(19) United States
(12) Patent Application Publication
(10) Pub. No.: US 2022/0145302 A1
(43) Pub. Date: May 12, 2022

(54) TARGETS AND METHODS FOR TREATING EPSTEIN-BARR VIRUS MEDIATED NEURODEGENERATION

(71) Applicant: Battle Biotech, LLC, Woodinville, WA (US)

(72) Inventor: Richelle Gayle Cutler, Oro Valley, AZ (US)

(21) Appl. No.: 17/093,436

(22) Filed: Nov. 9, 2020

(51) Int. Cl. C12N 15/113; C12N 15/63; C12N 15/86; A61K 31/65; A61K 31/7088; A61K 47/69; A61K 47/54

(52) U.S. Cl. CPC C12N 15/1133; A61K 48/00; C12N 15/86; A61K 31/65; A61K 31/7088; A61K 47/6911; A61K 47/549; C12N 2810/855; C12N 2310/11; C12N 2840/203; C12N 2310/321; C12N 2310/322; C12N 2310/3231; C12N 2310/315; C12N 2310/3515; C12N 2310/346; C12N 15/635

(57) ABSTRACT

Amyloid precursor protein (APP) dysfunction is a key feature in Alzheimer's disease (AD). The sortilin-related receptor 1 (SORLA) functions as a chaperone protein to APP and has reduced expression in AD brains. The APP and SORLA dysfunction results in homeostasis destabilization. Herpesviruses are suspected to be involved in AD pathogenesis. Using a strategic nucleotide BLAST to query SORL1 and APP nucleotide alignment on all Herpesviridae genomes identified similarity sequences from the Epstein-Barr virus and herpes simplex virus 2. The invention describes a treatment to alleviate EBV and HSV2-mediated neurodegeneration by delivering antisense oligonucleotides sequences that target the EBV and HSV2 non-coding sequences to block SORLA and APP disruption. The invention further describes methods to eradicate EBV infection by delivering inducible expression of antisense oligonucleotides targeting EBV genes or an inducible CRISPR/Cas gene-editing system, together with an expression construct encoding anti-apoptotic proteins or with anti-apoptotic proteins for the prevention of cell-mediated apoptosis.

### SEQUENCE LISTING

The sequences are disclosed in an 872 Kb text file named 10004B-US-NP-Sequences-as-filed, saved on Aug. 5, 2021, and are herein incorporated by reference.

### BACKGROUND

The age-corrected incidence of Alzheimer's disease (AD) per 100,000 is increasing. In 2000, the U.S. annual death rate from AD per 100,000 was 17.6, and in 2017 that number was 37.3. The rate of AD is expected to increase by 3-fold in the next 20 years. The baby boomer population bubble does not explain the rate increase, and while improved AD diagnosis may explain some increase, it cannot explain it all. AD is a complex disease that is caused by variants in host genetics, which change slowly over generations, and environmental factors, which change much faster. The increasing AD rate must be related to the increasing pervasiveness of an environmental factor.

The human herpesvirus 4, also known as the Epstein-Barr virus (EBV), could be an environmental driver behind the increasing AD prevalence. EBV DNA is significantly higher in peripheral blood leukocytes from AD patients than age-matched control, p=0.002 (Carbone et al. 2014). Recently, the Wyss-Coray laboratory reported finding clonally expanded T cells targeting EBV antigens in brain lesions from AD patients (Gate et al. 2020). In 1992, it was reported that EBV could transform B cell lines isolated from AD patients significantly more efficiently than B cells from healthy controls (Ounanian et al. 1992). This finding suggests that EBV entry receptor allotypes can increase EBV infection vulnerability which may increase AD risk. The EBV entry receptors are CD21 (CR2), CD35 (CR1), EphA2, integrin receptors (αvβ5, αvβ6, αvβ8), NRP1, NMHC-IIA, and HLA-DRB1.

Individuals express different EBV entry receptor alleles and EBV strain variants have different cell tropism. Together, strain and host genetic variability contribute to disease expression. Consistent with a role for EBV in AD, CD35, and HLA-DRB1 gene variants are significantly associated with AD risk (Chung et al. 2014; Lu et al. 2017). Notably, individuals with HLA-DRB1 antigen 13 have increased EBV seropositivity (Jabs et al. 1999), while healthy women harboring a single nucleotide variant, HLA-DRB1*13:02, have stable gray matter volume with age compared to a woman that does not (James et al. 2018).

Endothelial cells express CD21 and CD35 (Timens et al. 1991; Collard et al. 1999). Interestingly, EBV reportedly infects primary human brain microvessel endothelial cells in culture (Casiraghi et al. 2011; Jones et al. 1995). An MRI from an EBV encephalitis case showed inflammation restricted to brain microcirculation (Di Carlo et al. 2011); other case reports affirm that EBV infection is associated with cerebral vasculopathy (Weeks et al. 2006). In 1997, Miyakawa reported electron microscopy findings on vascular pathology in AD brains and emphasized the “disturbance of microvessels” (Miyakawa 1997).

Coculturing neurons with microvessels or culturing neurons with microvessel-conditioned media kills neurons when the microvessels are derived from AD patients but not when the microvessels are from healthy cognitive controls (Grammas et al. 2011). Another finding that highlights microvessels is that angiogenesis is significantly upregulated in the hippocampus of AD brains, resulting in increased vascular density (Desai et al. 2009). Interestingly, EBV proteins and miRNAs promote angiogenesis (Rivera-Soto and Damania 2019).

Erythrocytes express CD35 and are anomalous in AD patients (Kosenko et al. 2017). Furthermore, receiving washed erythrocyte cell transfusions significantly increases AD risk (Lin et al. 2019). Interestingly, immunolabeling for CD35 in the brain is mostly restricted to astrocytes (Fonseca et al. 2016). Since EBV has also been found to infect neurons, EBV is likely to bind other cell-surface receptors on neurons besides CD35 (Jha et al. 2015).

EBV is involved in the pathogenesis of multiple sclerosis, for which myelin loss is the cardinal pathology (Mechelli et al. 2015). Myelin degeneration also occurs in AD, and multiple sclerosis and AD coexist in some patients (Luczynski et al. 2019). Multiple sclerosis is associated with increased amyloid precursor protein expression and amyloid-β deposition (Chandra 2015). Coincidently, EBV cancers can produce amyloid-β deposits, and the amyloid precursor protein is upregulated in EBV nasopharyngeal carcinomas and lymphomas (Khan et al. 2019; Lai and Tay 2016; Nassif and Ozdemirli 2013).

AD is associated with decreased numbers of professional antigen-presenting dendritic cells (Ciaramella et al. 2016), and acute EBV infection is associated with reduced dendritic cell number (Panikkar et al. 2015). AD is associated with significantly elevated human IL-10 levels resulting in reduced immune activity (D'Anna et al. 2017), and EBV encodes and upregulates expression of a viral IL-10 homolog (Jog et al. 2018). Furthermore, immunosuppression by coinfection with other herpesviruses is likely to increase the risk of EBV-mediated neurodegeneration.

EBV infection is associated with diseases that are associated with dementia. For example, EBV infections are considered causal to Sjogren's syndrome, lupus erythematosus, and multiple sclerosis (Casiraghi et al. 2011; Croia et al. 2014; Parks et al. 2005) and each significantly increases the risk of dementia with age (Zhao et al. 2018; Chen et al. 2019; Luczynski et al. 2019; Hou et al. 2019). In addition, because the EBV multifunctional transcriptional activator EBNA2 binds to genetic risk variants associated with these autoimmune diseases (Harley et al. 2018), EBNA2 variants could be a determinant of EBV-mediated disease expression.

### SUMMARY

Alzheimer's disease (AD) is associated with amyloid-β deposits originating from amyloid precursor protein (APP) misprocessing. APP dysfunction can have diverse destabilizing effects. The sortilin-related receptor 1 (SORLA) assists in APP transport and is reduced in the AD brain. A BLAST search identified Epstein-Barr virus (EBV) non-coding RNAs (ncRNAs) with sequence similarity to SORL1 and APP intron sequences. The invention describes single-stranded RNA or DNA oligonucleotides that prevent EBV non-coding RNAs from binding to SORL1 or APP human sequences, wherein the oligonucleotide comprises at least 7 contiguous nucleotides that may be bidirectional and at least 70% identical to any sequence from SEQ ID NOs: 3-24. The invention also describes methods of treating EBV-mediated neurodegeneration by delivering RNA or DNA encoding oligonucleotides complementary to EBV non-coding RNA, wherein the oligonucleotide comprises at least 7 contiguous nucleotides that may be bidirectional and at least 70% identical to any sequence from SEQ ID NOs: 3-24. The oligonucleotides are modified to enhance affinity and nuclease resistance. Oligonucleotides may be delivered complexed in a lipid nanoparticle or encapsulated with a liposome. The oligonucleotide, lipid nanoparticle, or liposome may be conjugated with a targeting moiety. In some embodiments, the oligonucleotides are inserted in an expression construct within a viral vector and delivered by in vivo transduction.

The invention further describes a method for treating EBV-infection by delivering a DNA or RNA construct encoding antisense oligonucleotides targeting EBV genes, wherein the oligonucleotide sequences are 70% identical to any contiguous sequences from SEQ ID NOs: 27-146. The oligonucleotide expression construct is controlled by an inducible reverse-tetracycline-transactivator transcription domain (SEQ ID NO: 148), wherein the antisense oligonucleotide construct is expressed when a patient is administered doxycycline. In some instances, multiple oligonucleotides are co-expressed using intervening internal ribosome entry sites with an integrated ATG start codon. In one embodiment, the inducible oligonucleotide construct is delivered with a construct encoding the tetracycline-transactivator (SEQ ID NO: 147) and a BCL-2 family member protein (SEQ ID NOs: 149-154) or BIRC5 (SEQ ID NO: 155). The anti-apoptotic gene is co-expressed with the transactivator on a bicistronic message using an internal ribosome entry site. Co-expression ensures anti-apoptotic expression with the transactivator. In some instances, the nucleotide sequences are modified for codon optimization. In some embodiments, the inducible oligonucleotide construct and anti-apoptotic construct are encapsulated in a lipid nanoparticle or liposome. In some embodiments, the expression constructs are inserted into a viral vector for delivery by transduction. In some embodiments, the oligonucleotides (SEQ ID NOs: 27-146) are delivered together with the anti-apoptotic proteins (SEQ ID NOs: 157-163) in liposomes.

The invention further describes a method for treating EBV-infection by delivering a CRISPR/Cas gene-editing system comprising a Cas nuclease that is programmed by multiple guide strands comprising flanking ends of EBNA1 (SEQ ID NOs: 165-166), repeat regions (SEQ ID NOs: 167-168), 5′ exon of EBNA2 (SEQ ID NOs: 169-170), and the 5′ exon of latent membrane protein-1 (SEQ ID NOs: 171-172), wherein the guide strand sequences target Cas nuclease cutting to complementary EBV sequences to eliminate the EBV genome. A Cas nuclease (SEQ ID NO: 173) and one or more guide strands are encoded in a construct controlled by an inducible reverse tetracycline-transactivator transcription domain. In some instances, the guide strand is controlled separately with a U6 promoter (SEQ ID NO: 174) or minimal cytomegalovirus promoter (SEQ ID NO: 175) and cytomegalovirus enhancer (SEQ ID NO: 176). The CRISPR/Cas construct is delivered together with a construct encoding the tetracycline-transactivator and a BCL-2 family member protein (SEQ ID NOs: 157-162) or BIRC5 (SEQ ID NO: 163) to prevent cell-mediated death. The anti-apoptotic gene is co-expressed with the transactivator by using an internal ribosome entry site. In some embodiments, the constructs are delivered complexed within a lipid nanoparticle or encapsulated in a liposome, which may be conjugated to a targeting moiety. In some embodiments, expression constructs are inserted into a viral vector for delivery by transduction. The described therapeutics could be administered by intracranial, intravenous, intradermal, subcutaneous, or intramuscular injection.

### BRIEF DESCRIPTION OF THE DRAWINGS

FIG. 1. Depicts the locations of three EBV similarity sequences (SEQ ID NO: 10) in the SORL1 gene (SEQ ID NO: 2).

FIG. 2. Depicts the location of two EBV similarity sequences (SEQ ID NO: 10) in the APP gene (SEQ ID NO: 1).

FIG. 3. Depicts the sequence (SEQ ID NO: 25) from EBV isolate H002213 containing putative EBV ncRNA complementary to SORL1 (SEQ ID NO: 2).

FIG. 4. Depicts the location of the 21-nucleotide EBV sequence similarity (SEQ ID NO: 24) in intron 32 of SORL1 (SEQ ID NO: 2).

FIG. 5. Depicts the locations of the EBV-SORL1 similarity sequence (SEQ ID NO: 24) in the EBNA-LP gene.

### DETAILED DESCRIPTION

Abnormal tau phosphorylation and neurofibrillary tangles appear decades before AD symptoms in cognitively healthy adults. Neurofibrillary tangles are first apparent in the entorhinal cortex and the adrenergic neurons of the locus coeruleus (LC) that project to the entorhinal cortex (Braak and Braak 1991; Tsartsalis et al. 2018). Neurofilament aggregation could begin when EBV infects the neurovascular unit of the transentorhinal cortex, including the endothelial cells, smooth muscle cells, pericytes, and astrocytes. EBV could spread from the vasculature to the innervating adrenergic neurons from the LC.

The adrenergic system is dysfunctional in AD patients (Gannon et al. 2015). Adrenergic neurons in the LC project axons throughout the brain to control optimal circadian homeostatic activity through the release of norepinephrine and coordinated peptides. Adrenergic neurons can control activity by regulating vascular tone. Adrenergic neuron axon terminals release norepinephrine onto smooth muscle cells in small cerebral arterials and onto pericytes in microvessels to induce vasoconstriction or vasodilation. Norepinephrine binds α-adrenergic receptors on vessels to induce vasoconstriction and binds β-adrenergic receptors to induce vasorelaxation. Age-related loss of estrogen reduces β₁- and β₃-adrenergic receptor expression resulting in reduced vasodilation (Riedel et al. 2019). Thus, the age-related loss of estrogen can reduce vasodilation in women. Accordingly, more women have higher blood pressure (Abramson et al. 2018), and vasoconstriction increases by two-fold in ovariectomized rats.

Amyloid-β deposits are a key pathological feature in AD brains. Increased amyloidogenesis in microvessels increases the expression of inflammatory cytokines, which leads to prothrombotic protein expression, such as increased tissue factor and decreased thrombin expression. Approximately 80% of AD patients have some degree of cerebral amyloid angiography (CAA) characterized by amyloid-β deposits in the vessel wall of small cortical arterials (Brenowitz et al. 2015). CAA narrows the vascular lumen and is associated with hemorrhagic bleeds, microinfarcts, and white matter pathology. Amyloid-β aggregates are also found among degenerating microcapillaries (Miyakawa 1997). The amyloid-associated vascular pathology can result in ischemic brain injury and lead to cognitive impairment.

Amyloid-β deposits may promote the coagulation factor XII pathway to activate the protease thrombin (Zamolodchikov et al. 2016). Thrombin cleaves fibrinogen molecules releasing fibrin to form an insoluble fibrous clot. AD and EBV are associated with high fibrinogen and fibrin levels (van Oijen et al. 2005; Cortes-Canteli et al. 2019; Tang et al. 2014). In addition to amyloid-β, EBV antigens exposed on the vascular surface can activate the complement system, including the C3a and C5a fragments that are unique to the AD blood proteome (Baird et al. 2015). Complement activation increases neutrophil attachment and aggregation, occluding microvessels. Occluded microvessels degenerate, resulting in local hypoxia. In a vicious cycle, hypoxia can further amplify amyloid-β processing (Bennett et al. 2000) and increase CD35 cell surface expression, which increases EBV entry efficiency (Collard et al. 1999).

Thrombosis in microvessels can cause silent inicroinfarcts. Reduced cerebral blood perfusion is an early sign of AD (Korte et al. 2020). The number of microinfarcts in brains from AD patients could be in the hundreds to thousands but are currently only visible in high field strength MRI, although infarcts smaller than 100 microns remain invisible (Reijmer et al. 2016). Similarly, EBV infection of endothelial cells in the glymphatic and meningeal lymphatic systems could block the drainage of metabolic waste.

Chronic hypoxia can cause inflammation and cell death. Inflammatory signaling activates the renin-angiotensin system, which can also increase vasoconstriction (Satou et al. 2018). Hypoxia can cause abnormal tau hyperphosphorylation, and entorhinal cortical cells are highly sensitive to hypoxia (Kirino et al. 1984). A mitigating multigenic response to hypoxia is mediated by the release of the hypoxia-inducible factor-alpha 1 (HIF-1). However, the HIF-1 response may be dysfunctional in elderly humans since HIF-1 binding to hypoxia-response element is deficient in senescent mice (Frenkel-Denkberg et al. 1999).

Sortilin-related receptor 1 (SORL1) and amyloid precursor protein (APP) gene variants influence AD risk. The SORL1 protein, SORLA, associates with APP at its C-terminal end, restricting APP location to the Golgi or cell membrane. Without SORLA, APP moves through the endosomal pathway where it is cleaved to amyloid-β by β- and γ-secretase. Norepinephrine-mediated activation of α₂-adrenergic receptors changes APP localization by disrupting SORLA and APP binding (Chen et al. 2014). Once disassociated, APP can bind activated α₂-adrenergic receptors, stabilizing the α₂-adrenergic receptor at the cell surface, thereby preventing internalization and permitting inhibitory feedback desensitization (Zhang et al. 2017). Without APP localization to activated α₂-adrenergic receptors, the α₂-adrenergic receptors bind arrestin and are internalized. The internalization of presynaptic inhibitory α₂-adrenergic receptors desensitizes feedback control, increasing norepinephrine-mediated release. Excess norepinephrine released at vascular innervation is a potent attractant for EBV infected B cells, monocytes, and macrophages expressing β₂-adrenergic receptors.

The α₁- and α₂-adrenergic receptors do not share amino acid sequence similarity in the intracellular domain that associates with APP. Thus, the loss of APP causes α₂-adrenergic receptor internalization, whereas the α₁-adrenergic receptor is unaffected. Persistent norepinephrine-mediated α₁-adrenergic receptor signaling can result in chronic vasoconstriction. The loss of norepinephrine-mediated α₂-adrenergic receptor vasoconstriction could explain the dysfunctional baroreflex in AD patients.

Astrocytes express the EBV entry receptors CD35 (CR1) and EphA2, and α₁-, α₂-, β₁- and β₂-adrenergic receptors. Norepinephrine activated β₂-adrenergic receptor is associated with increased amyloidogenesis (Ni et al. 2006). Amyloid-β reportedly acts as an allosteric ligand to enhance adrenergic signaling (Nortley et al. 2019; Zhang et al. 2020). Accordingly, in AD brains amyloid-βimmunoreactivity is found in astrocytes, not neurons (Kurt et al. 1999). Increased norepinephrine- and amyloid-β mediated α₁-adrenergic receptor signaling stimulates glutamate and ATP release from astrocytes. Activation of α₂-adrenergic receptors upregulates calcium oscillations in astrocytes, which increases the release of the inhibitory neurotransmitter GABA. GABA in turn reduces calcium oscillation in neurons (Gaidin et al. 2020). However, EBV-mediated APP disruption would decrease α₂-adrenergic receptor stabilization at the astrocyte cell surface resulting in less norepinephrine-mediated GABA release. The loss of norepinephrine-mediated astrocytic GABA release may dysregulate diurnal neuronal activity. Reduced astrocytic GABA release could explain why the astrocyte GABA transporter (BGT-1) is significantly increased in the AD hippocampus (Fuhrer et al. 2017).

Neurons and astrocytes express the ionotropic NMDA receptor (NMDAR). Amyloid-β can bind to NMDARs and activate calcium release (Li et al. 2009). In neurons, sustained intercellular calcium can induce long-term depression, leading to neurite loss (Sheng and Erturk 2014). A higher intercellular calcium concentration increases intracellular chloride. Increased intracellular chloride means that GABA-activated chloride channels result in chloride efflux and depolarization. This situation resembles development, wherein GABA from inhibitory interneurons acts as an excitatory neurotransmitter, stimulating neurite outgrowth, which could explain abnormal neurite outgrowth surrounding amyloid-β deposits in the AD brain.

The activation of α₁- and α₂-adrenergic receptors on neurons reduces the NMDAR-mediated excitatory postsynaptic potentials (EPSPs) amplitude but not the paired-pulse response (Liu et al. 2006). Amyloid-β oligomers can bind allosterically to α₂-adrenergic receptors to enhance norepinephrine-mediated G-protein activation (Zhang et al. 2020). The decreased regulator of G-protein signaling 2 (RGS2) gene expression found in AD brain tissue would potentiate decreased NMDAR current. Thus, increased norepinephrine- and amyloid-β-mediated α₁- and α₂-adrenergic receptor activation could reduce neuronal EPSP amplitudes, which could affect synaptic maintenance.

APP and SORLA are also important in regulating neuron survival. APP regulates activation and localization of the nerve growth factor (NGF) receptor, tropomyosin receptor kinase A. SORLA regulates the trafficking of the brain-derived neurotrophic factor (BDNF) receptor, tropomyosin receptor kinase B. Significant neuropathology can result from the disruption of APP and SORLA expression (Barthelson et al. 2020). Thus, targeting EBV factors disrupting SORLA and APP disruption is critical to reducing EBV-mediated AD pathology.

There is evidence that APP has anti-microbial properties (Kumar et al. 2016; Eimer et al. 2018). In HIV, APP associates with the HIV Gag polyprotein, wherein APP binding restricts HIV particles from translocating to lipid rafts, but Gag induces APP secretase cleavage for release, resulting in amyloidogenic processing (Chai et al. 2017; Hategan et al. 2019). If a herpesvirus factor interacts with APP it could have sequence similarity to APP (SEQ ID NO: 1). Using GenBank's Basic Local Alignment Search Tool (BLAST) to search for sequence similarity between SORL1 (SEQ ID NO: 2) and the Herpesviridae family identified SORL1 sequences in EBV and human herpes simplex virus 2 (HSV2) as shown in table 1 (SEQ ID NOs: 3-14). The highest sequence similarity targeted the C-terminal end of SORL1, with an Expect value of 5×10⁻¹¹⁶, matching 610/634 nucleotides. The same GenBank BLAST found 10 EBV isolates with 35-nucleotide sequence similarity to SORL1 (SEQ ID NO: 10) and an HSV2 isolate with 48-nucleotide sequence similarity to SORL1 (SEQ ID NO: 7).

TABLE 1
Human herpesvirus strains and isolates with sequence similarity to human SORL1 gene

| | HHV isolate | | Percent | Virus | Virus | SORL1 | SEQ ID | |
|---|---|---|---|---|---|---|---|---|
| HHV type-isolate | Accession | E value | identity | Position | region | region | No. | Similarity Sequence |
| HHV 4-HKHD40 | MH590409.1 | 5.00E-116 | 96 | 158635 | intergenic | intron | 3 | GTTAGTTACATATGTATACATGTGCCATGNTGGTG... |
| HHV 4-HKNPC60 | MH590571.1 | 5.00E-116 | 99 | 36456 | intergenic | intron | 4 | GCCGCAATAAACATACGTGTGCATGTGTCTTTATA... |
| HHV 4-Namalwa | AH002364.2 | 4.00E-51 | 91 | 412 | repeat | intron | 5 | CCTGTAGTCCCAGCTACTCAGGAGGCTGAGGCAG... |
| HHV 4-HKHD130 | MH590499.1 | 7.00E-16 | 87 | 37475 | intergenic | intron | 6 | AATAGGCTGNGTGCGGTGGCTCACACCTGTAATN... |
| HHV 2-2006-16150CAM | MH790658.1 | 1.00E-07 | 92 | 11357 | intergenic | intron | 7 | ACATTAGGTATATCTCCTAATGCTATCCCTCCCCC... |
| HHV 4-HKHD73 | MH590442.1 | 3.00E-07 | 83 | 95948 | intergenic | intron | 8 | GCACTCCAGCCTGGGCAACAGAGTGAGACCCTAAC... |
| HHV 4-HKHD7 | MH590376.1 | 1.00E-06 | 67 | 36674 | intergenic | intron | 9 | AATTTTGTTGATCTTTTCAAAAAACCAGCTCCTGG... |
| HHV 4-H002213 | KP968264.1 | 2.00E-04 | 97 | 159585 | A73 intron | intron | 10 | CTGCACTCCAGCCTGGGCAACAGAGCGAGACCCTG |
| HHV 4-VGO | KP968260.1 | 2.00E-04 | 97 | 159392 | A73 intron | intron | 10 | CTGCACTCCAGCCTGGGCAACAGAGCGAGACCCTG |
| HHV 4-SCI | KP968259.1 | 2.00F-04 | 97 | 15957 | A73 intron | intron | 10 | CTGCACTCCAGCCTGGGCAACAGAGCGAGACCCTG |
| HHV 4-CCH | KP968257.1 | 2.00E-04 | 97 | 159504 | A73 intron | intron | 10 | CTGCACTCCAGCCTGGGCAACAGAGCGAGACCCTG |
| HHV 4-VA | KT001102.1 | 2.00E-04 | 97 | 159547 | A73 intron | intron | 10 | CTGCACTCCAGCCTGGGCAACAGAGCGAGACCCTG |
| HHV 4-FNR | KR063345.1 | 2.00E-04 | 97 | 159600 | A73 intron | intron | 10 | CTGCACTCCAGCCTGGGCAACAGAGCGAGACCCTG |
| HHV 4-H03753A | KR063342.1 | 2.00E-04 | 97 | 159580 | A73 intron | intron | 10 | CTGCACTCCAGCCTGGGCAACAGAGCGAGACCCTG |
| HHV 4-K4123-MiEBV | KC440852.1 | 2.00E-04 | 97 | 159589 | A73 intron | intron | 10 | CTGCACTCCAGCCTGGGCAACAGAGCGAGACCCTG |
| HHV 4-K4123-Mi | KC440851.1 | 2.00E-04 | 97 | 159567 | A73 intron | intron | 10 | CTGCACTCCAGCCTGGGCAACAGAGCGAGACCCTG |
| HHV 4-B95-8 | AJ507799 | 3.00E-04 | 94 | 148162 | A73 intron | intron | 10 | CTGCAGTCCTGCCTGGCGCAACAGAGCGAGACCCTG |
| HHV 4-RPF | KR063344.1 | 6.00E-04 | 100 | 159601 | A73 intron | intron | 11 | GTCTCGCTCTGTTGCCCAGGCTGGACTGCAG |
| HHV 4-HKHD95 | MH590464.1 | 8.00E-03 | 94 | 36494 | intergenic | intron | 12 | GGCTAATTTTTTTGTATTTTTAGTAGAGATGGGG |
| HHV 4-HKHD141 | MH590510.1 | 1.30E-02 | 94 | 96579 | intergenic | intron | 13 | TTCTCCTGCCTCAGCCTCCNGAGTAGCTGGGATT |
| HHV 4-HKHD27 | MH590396.1 | 2.60E-02 | 97 | 27439 | intergenic | intron | 14 | CCATCTTGGCTCACTGCAACCTCCACCTCCC |

A BLAST search for sequence similarity between APP (SEQ ID NO: 1) and the Herpesviridae family identified some of the same similarity sequences as between SORL1 (SEQ ID NO: 2) and EBV and human herpes simplex virus-2 (HSV2) (Table 2). Some EBV isolates contain long sequences of APP intron alignment. For instance, isolates HKNPC60 and HKHD40 contain 2,486 and 2,357 nucleotides, respectively, aligned with 97% ungapped similarity to APP intron 13. The Namalwa EBV cell line contains an 82-nucleotide sequence with 93% similarity to APP intron 17.

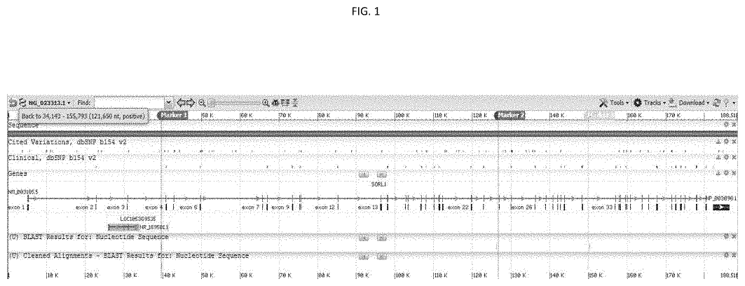

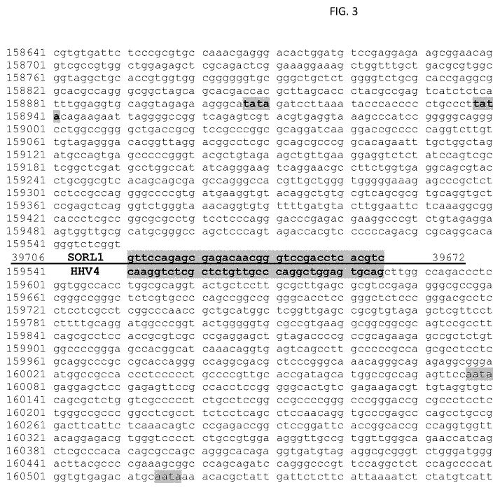

The 35-nucleotide sequence (SEQ ID NO:10) matching APP and SORL1 is from the same EBV location (159362-159601) located in intron 3 of A73. In SORL1 and APP, the sequence targets exclusively introns on both strands. In SORL1, the antisense sequence targets intron 3 (39672) and intron 23 (126323) just 5′ from exon 24, while a sense alignment is in intron 32 (FIG. 1). Table 1 lists SEQ ID NO: 10 in plus/minus alignment, which is in the 3′ to 5′ direction or antiparallel and complementary to the human herpesvirus 4 (HHV4) sequence as shown in FIG. 3. The sequence in intron 3 is 1109-nucleotides 5′ from exon 4 (40781) and 664-nucleotides 3′ from a clinical SNP (rs11600875) at 39008 (Reynolds et al. 2013; McCarthy et al. 2012).

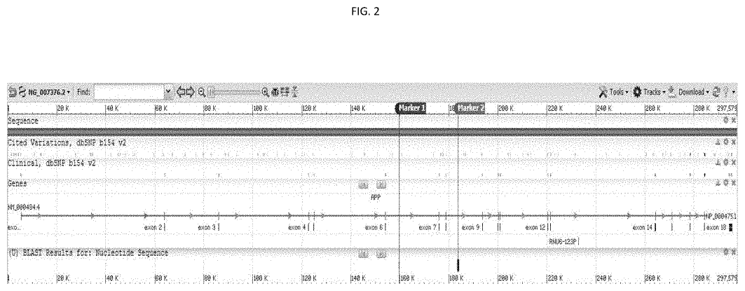

In APP, SEQ ID NO: 10 maps to intron 6 (183750) and intron 8 (159501) (FIG. 2). In EBV, two transcription start sites (TATA) are located 644 and 613 nucleotides 5′ of the complement, which predicts a non-coding RNA (ncRNA) of a least 644 or 613 nucleotides (FIG. 3). EBV may also transcribe an ncRNA complement from 3′ to 5′ that aligns with the intron 32 sequence and use the TATT at 160077 or 160518, which would produce at least a 502 or 943 nucleotide sequence. The 1920 EBV nucleotide sequence bracketing the complement to SEQ ID NO: 10 is encoded in SEQ ID NO: 25. A BLAST search with SEQ ID NO: 10 against Homo sapiens genomic and RNA sequences identified other RNA sequence similarities in sense, such as the Zinc Finger protein 677 mRNA and TMEM241 exon1 ncRNA.

TABLE 2
Human herpesvirus strains with sequence similarity to the human APP gene

| | HHV isolate | | Percent | Virus | Virus | APP | SEQ ID | |
|---|---|---|---|---|---|---|---|---|
| HHV type-isolate | Accession | E value | identity | Position | region | region | No. | Similarity Sequence |
| HHV 4-HKNPC60 | MH590571.1 | 1.00E-148 | 97 | 36456 | intergenic | intron | 4 | GCCGCAATAAACATACGTGTGCATGTGTCTTT... |
| HHV 4-HKHD40 | MH590409.1 | 2.00E-132 | 97 | 158638 | intergenic | intron | 15 | CAGGTTAGTTACATATGTATACATGTGCCATG... |
| HHV 4-HKHD7 | MH590376.1 | 5.00E-123 | 98 | 36670 | intergenic | intron | 16 | TATCAATTTTGTTGATCTTTTCAAAAAACCA... |
| HHV 4-Namalwa | AH002364.2 | 6.00E-51 | 89 | 412 | repeat | intron | 5 | CCTGTAGTCCCAGCTACTCAGGAGGCTGAG... |
| HHV 4-HKHD130 | MH590499.1 | 5.00E-14 | 83 | 37423 | intergenic | intron | 17 | GCGGTGGCTCACGCCTGTAATCCCAGCACTT... |
| HHV 4-HKHD73 | MH590442.1 | 6.00E-13 | 83 | 96041 | EBNA-1 | intron | 18 | ...GTCTCACTCTGTTGCCCAGGCTGGAGTGC |
| HHV 2-2006-16150C | MH790658.1 | 2.00E-07 | 92 | 11358 | intergenic | intron | 19 | CATTAGGTATATCTCCTAATGCTATCCCTCC... |
| HHV 6-HP36C6 | KY315533.2 | 8.00E-05 | 84 | 82980 | intergenic | intron | 20 | TTGTTTTAAGCCAATCTGTTTGTGATACTTTG... |
| HHV 4-HKHD141 | MH590510.1 | 1.00E-04 | 95 | 96579 | intergenic | intron | 21 | TTCTCCTGCCTCAGCCTCCNGAGTAGCTGGGA.. |
| HHV 4-HKHD141 | MH590510.1 | 1.00E-04 | 97 | 96546 | intergenic | intron | 22 | AATCCCAGCTACTCNGGAGGCTGAGGCAGGA... |
| HHV 4-HKHD95 | MH590464.1 | 0.001 | 97 | 36461 | intergenic | intron | 23 | GGCTAATTTTTTTGTATTTTTAGTAGAGATGGG.. |
| HHV 4-H002213 | KP968264.1 | 0.001 | 94 | 159586 | A73 intron | intron | 10 | CTGCACTCCAGCCTGGGCAACAGAGCGAGAC |
| HHV 4-VGO | KP968260.1 | 0.001 | 94 | 159362 | A73 intron | intron | 10 | CTGCACTCCAGCCTGGGCAACAGAGCGAGAC |
| HHV 4-SCL | KP968259.1 | 0.001 | 94 | 159573 | A73 intron | intron | 10 | CTGCACTCCAGCCTGGGCAACAGAGCGAGAC |
| HHV 4-CCH | KP968257.1 | 0.001 | 94 | 159535 | A73 intron | intron | 10 | CTGCACTCCAGCCTGGGCAACAGAGCGAGAC |
| HHV 4-VA | KT001102.1 | 0.001 | 94 | 159548 | A73 intron | intron | 10 | CTGCACTCCAGCCTGGGCAACAGAGCGAGAC |
| HHV 4-FNR | KR063345.1 | 0.001 | 94 | 159601 | A73 intron | intron | 10 | CTGCACTCCAGCCTGGGCAACAGAGCGAGAC |
| HHV 4-H03753A | KR063342.1 | 0.001 | 94 | 159581 | A73 intron | intron | 10 | CTGCACTCCAGCCTGGGCAACAGAGCGAGAC |
| HHV 4-K4123-MiEBV | KC440852.1 | 0.001 | 94 | 159590 | A73 intron | intron | 10 | CTGCACTCCAGCCTGGGCAACAGAGCGAGAC |
| HHV 4-K4123-Mi | KC440851.1 | 0.001 | 94 | 159568 | A73 intron | intron | 10 | CTGCACTCCAGCCTGGGCAACAGAGCGAGAC |
| HHV 4-RPF | KR063344.1 | 0.003 | 94 | 159631 | A73 intron | intron | 10 | CTGCAGTCCAGCCTGGGCAACAGAGCGAGAC.. |
| HHV 4-HKHD27 | MH590396.1 | 0.006 | 100 | 27469 | intergenic | intron | 24 | GGGAGGTGGAGGTTGCAGTGAGCCAAGAT |

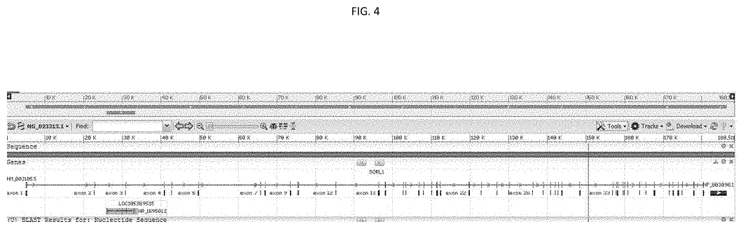

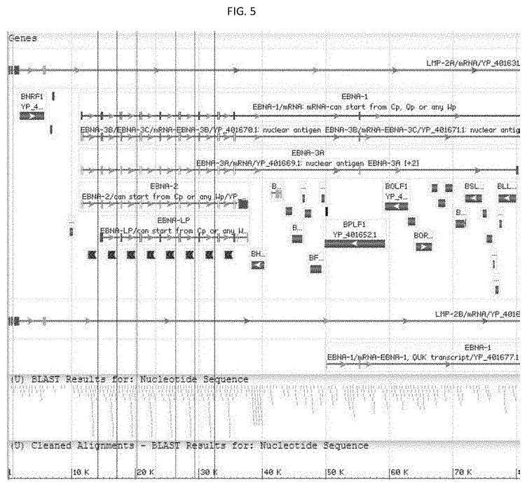

The Virus Pathogen Database Analysis Resource (ViPR) contains 1837 complete Herpesviridae genomes. A BLAST using this database returned essentially the same EBV sequence similarity as the GenBank BLAST for SORL1 and APP. A restrictive BLAST of only EBV strains identified one 21-nucleotide sequence (SEQ ID NO: 24) with similarity to SORL1 that maps to intron 32 in SORL1 with an Expect value of 4×10⁻⁷ (FIG. 4). In the EBV isolate AJ507799.2, the 21-nucleotide sequence (SEQ ID NO: 24) locates to multiple EBNA-LP introns, 3′ of the BWRF1 repeats (FIG. 5). The 21-nucleotide sequence (SEQ ID NO: 24) has no significant alignment to APP but does have antisense alignment with the gene POU class 2 homeobox 3 gene (POU2F3). Interestingly, a KEGG pathway map shows POU2f3 functionally equivalent to Oct-1, which regulates human herpes simplex virus-1 (HSV1) immediate early gene transcription. In EBV, Oct-1 enhances BRLF1-mediated lytic replication.

An estimated 13% of herpesvirus proteins show sequence similarity to human genes, and about 54% of these sequences interact functionally with the host (Holzerlandt et al. 2002). Thus, the EBV sequences may not affect human SORL1 or APP expression. However, it is curious that only EBV, except for one alignment in HSV2, shows the same 31/35 nucleotide sequence similarity with SORL1 and APP. The functionality of the identified similarity sequences could be easily tested by measuring amyloid-β production in EBV infected endothelial, smooth muscle, and astrocytic cell lines transfected with test or scrambled control oligonucleotides.

In one embodiment, the oligonucleotides (SEQ ID NOs: 3-6; 8-24), sense or antisense or antiparallel, are delivered in vivo to block EBV-mediated SORLA and APP disruption. The EBV ncRNAs could target SORL1 and APP functional splicing elements to produce exon skipping or premature termination. For instance, the cis-natural antisense ncRNA 51A maps to intron 1 of SORL1 and reduces the expression of the dominant SORL1 splice variant A, leading to increased amyloidogenesis (Ciarlo et al. 2013). The 21-nucleotide sequence (SEQ ID NO: 24) could disrupt the splicing of the SORL1 exon coding for the last LDLR class A 11 complement repeat domain and the downstream exons that code for sites interacting with SORLA shuttling proteins. Alternatively, the EBV ncRNA binding could suppress SORL1 transcription.

Neurons from AD brains show abnormal expression of cell cycle entry proteins cyclins-D and —B and increased hyperploidy (Yang et al. 2003; Frade and López-Sánchez 2017). Cell cycle entry is abnormal for postmitotic neurons and is linked to synaptic dysfunction and neuron death (Herrup 2010; Barrio-Alonso et al. 2018). EBV expresses high levels of two short ncRNA molecules, EBER1 and EBER2. EBER expression induces IL-6 mediated activation of signal transducers and activators of transcription 3 (STAT3). STAT3 activation decreases the expression of cyclin-dependent kinase inhibitors p21 and p27, which releases cyclin-dependent kinases 2 and 4 (CDK4 and CDK2) inhibition, allowing cyclins to promote GUS transition (Yin et al. 2019; Yajima et al. 2005). CDK4 activation induces cell death by hyperphosphorylation of the pRb family member p130. Phosphorylated p130 binds chromatin modifiers Suv39H1 and HDAC1, releasing the transcription factor E2F4, which binds transcription factors B and C-Myb promoters to initiate transcription. Transcription factors B and C-Myb bind the promoter of the proapoptotic BH3-containing Bim to initiate transcription. Bim activates BAX/BAK, which forms multimeric pores in the mitochondrial membrane and releases cytochrome c. Cytochrome c binds the protein 14-3-3ε to release its inhibition over the apoptotic protease activating factor-1, allowing apoptosome formation and apoptosis-inducing caspase 9/3 activation (Greene et al. 2007). Accordingly, the upregulation of BIM expression in the AD brain compared to healthy control brain could in part be caused by EBER1 (Biswas et al. 2007).

EBER1 and EBER2 are found in extracellular vesicles adjacent to nasopharyngeal tumors (Cheng et al. 2019) and EBER1 was found secreted bound to the lupus La protein (Iwakiri et al. 2009). EBER1 was also found bound to L22 ribosomal protein (EBER-associated protein) in uninfected cells (Toczyski et al. 1994). The entry of EBER1 into uninfected neurons could induce cyclin-dependent kinase-mediated apoptosis. Moreover, EBERs binding to TLR3 in neurons can cause irreversible growth cone collapse and inhibit neurite outgrowth. Blocking extracellular EBER1 passage to neurons could prevent neuron dysfunction. The EBERs could also produce inflammation in microvessels or lymphatic vessels. Thus, in one embodiment, the oligonucleotides antisense to EBER1 (SEQ ID NOs: 27-36) and EBER2 (SEQ ID NOs: 37-46) are delivered in vivo to block EBER function.

The oligonucleotides described in SEQ ID NOs: 3-24 function by binding to complementary sequences to block DNA transcription by triplex-forming oligonucleotide, splicing, or ncRNA function. In some cases, the delivered oligonucleotide SEQ ID NOs: 3-6; 8-24 is antisense or complementary and antiparallel to the EBV ncRNA sequence and functions as an RNA or ribonucleoprotein sponge.

In another embodiment, locked-nucleic acid-modified nucleotides are included within the oligonucleotide. In some instances, nucleotides are modified with a 2′-O-methyl, 2′-O-Methoxyethyl, 2′-fluorine at 2′-ribose (OH), and 2′-fluoroarabinonucleic acid to increase annealing affinity to complementary RNA. A locked nucleotide contains a methylene bridge between the 2′ and 4′ positions of the ribose and increases binding affinity. A phosphorothioate or amide nucleotide linkage increases RNase resistance.

In some embodiments, the oligonucleotides are complexed with nanoparticles or contained within liposomes. In some embodiments, a targeting moiety is conjugated directly to the oligonucleotides with SEQ ID NOs: 3-24, lipid nanoparticles, or liposomes. A targeting moiety increases the cellular uptake by the intended target cell and therefore, therapeutic efficacy. In some embodiments, CD21 expressing cells are targeted by conjugation with an antibody or single-chain antigen-binding variable domain fragment (FAB). Unlike most viral entry receptors, CD21 expression is stable or even upregulated after EBV infection, making CD21 an ideal target for delivering an EBV therapeutic (Ogembo et al. 2013). In some embodiments, CD20 expressing B cells are targeted by conjugation with an antibody or single-chain antigen-binding variable domain fragment (FAB). In some embodiments, only EBV infected cells are targeted by using an antibody or a single-chain antigen-binding variable domain fragment with affinity to the extracellular portion of LMP2A/B, or BILF1 protein expressed on the infected cell surface. U.S. Pat. Nos. 10,800,848; 10,787,519; 10,550,188; 10,487,149 disclose compositions comprising an antibody or binding fragment conjugated to polynucleic acid molecules. In some embodiments, oligonucleotides target αᵥβ₃, or αᵥβ₆ on the cell surface by conjugating the oligonucleotide with the high-affinity peptides such as cRDGyK (Tian et al. 2018; Tabata et al. 2008). In some instances, oligonucleotides are labeled with azide using 3-azidoproprionic acid and reacted with antibodies functionalized with a dibenzocyclooctyne (DBCO) click group (Wiener et al. 2020).

In another embodiment, the cell-penetrating peptide from HIV-Tat (GRKKRRQRRRPPQ) (SEQ ID NO: 26) or similar cationic penetrating peptide is fused to oligonucleotides, lipid nanoparticles, or liposomes to enhance delivery (Astriab-Fisher et al. 2002; Farrell et al. 2004). For example, a cell-penetrating peptide can be a bipartite amphipathic peptide, such as MPG, which is derived from the fusion peptide domain of HIV-1 gp4l and the NLS of SV40 large T antigen (Simeoni et al. 2003). In another embodiment, the oligonucleotides are inserted in an expression construct within a viral vector and delivered by in vivo transduction. The viral vector may be an adreno-associated virus or lentivirus (Körbelin et al. 2016).

Blocking EBV-mediated SORLA and APP dysfunction can prevent vasoconstriction, dysfunctional baroreflex, increased thrombosis, and complement-mediated lysis of microvessel, glymphatic, and meningeal lymphatic endothelial cells, decreased NGF and BDNF signaling, and dysfunctional calcium signaling. Blocking EBER ribonucleoproteins may prevent non-cell-autonomous effects in non-infected cells. However, EBV infection could still cause catastrophic neuron degeneration if EBV infected astrocytes fail to support neurons. For instance, astrocytes provide glucose, water, and electrolytes, a blood-brain barrier, neurovascular coupling, and prevent excessive glutamate and K⁺ ions accumulation. Thus, preventing AD may require eradicating EBV infection.

EBV lytic replication can be inhibited by the antivirals, acyclovir and penciclovir, and their more bioavailable analogs, valaciclovir and famciclovir, respectively. However, EBV factors (EBNA1-4, LMP1, and EBERs) are expressed during different stages of latency and they alter the expression of thousands of genes. Thus, EBV-mediated disease cannot be managed by the current antivirals, which only prevent lytic replication.

EBV genomes can be eliminated by targeting EBNA1 (Noh et al. 2016; van Diemen et al. 2016). Wang and Quake report that targeting multiple EBV proteins can efficiently eliminate EBV genomes (Wang and Quake 2014). However, eliminating EBV genomes results in the loss of multiple EBV factors that prevent cell-mediated apoptosis, thus, permitting apoptosis execution. The apoptosis of vascular cells or astrocytes could cause massive hemorrhaging or catastrophic neuron death, respectively. Cell-mediated apoptosis can be prevented by increasing the expression of anti-apoptotic factors.

TABLE 3
List of EBV targets for viral eradication by antisense
oligonucleotides

| SEQ ID | | |
|---|---|---|
| NO | Target | Details |
| 27-36 | EBER1 | Secreted and blocks PKR |
| 37-46 | EBER2 | Binds cellular PAX5 to regulate EBV |
| | | transcription |
| 47-56 | EBNA1 | Activates intracellular immunity and blocks |
| | | episome maintenance |
| 57-66 | EBNA2 | Blocks cellular transactivator |
| 67-76 | LMP1 | Prevents NF-kB activation |
| 77-86 | RZLF1 | Prevent viral gene expression required for |
| | | lytic replication |
| 87-96 | BPLF1 | Major cellular protein binding hub |
| 97-106 | BFLF2 | Major cellular protein binding hub |
| 107-116 | BALF4 | Major cellular protein binding hub |
| 117-126 | SM | Prevent binding and inactivation of SP100 |
| 127-136 | BRRF1 | Major cellular protein binding hub |
| 137-146 | BVRF1 | Major cellular protein binding hub |

Efficient EBV genome elimination requires targeting multiple EBV factors simultaneously in the same cell (Table 3). Integral to eliminating EBV is to prevent EBV from blocking intrinsic immunity factors, including the promyelocytic leukemia protein nuclear body components (PML-NBs) or nuclear domain 10 (ND10). EBNA1 is a critical protein involved in EBV episome maintenance, lytic replication, and preventing cellular immunity. EBNA1 and SM inhibit antiviral PML-NBs activity. Blocking EBNA1 in EBV-lymphoma cells induces EBV genome loss (Noh et al. 2016). Interferon signaling is required to mobilize and intensify PML-NB antiviral activity. Interferon signaling is muted by LMP1 and by EBER1 binding to the double-stranded RNA-dependent protein kinase (PKR).

The EBV BZLF1 is expressed as an immediate-early gene and functions as the lytic switch transactivator to initiate regulatory gene expression required for lytic replication. The EBV protein LMP1 will be silenced to prevent autophagy activation and the unfolded protein response, which phosphorylates the eukaryotic translation initiation factor 2 alpha (eIF2α) to block protein synthesis. The EBV protein EBNA2 is a multifunctional transcriptional activator altering the expression of thousands of genes. Similarly, BPLF1, BFLF2, BALF4, BVRF1, and BRRF1 are targets because they are major EBV protein interaction hubs with human genes (Calderwood et al. 2007). Silencing these EBV genes will restore cellular homeostasis (Toyama et al. 2018).

In another embodiment, the antisense oligonucleotides from any combination of SEQ ID NOs: 27-146 are delivered in an expression construct under the control of a reverse inducible tetracycline-transactivator transcription domain (SEQ ID NO: 147). The oligonucleotide construct is delivered together with a construct encoding the transactivator gene (SEQ ID NO: 148) and a BCL-2 family member, such as BCL-2, BCL-XL, MCL-1, BFL-1, BCL-W, and BCL2L10 (SEQ ID NOs: 149-154) and/or survivin (BIRC5) (SEQ ID NO: 155). The transactivator and anti-apoptotic gene are co-expressed using an internal ribosome entry site (SEQ ID NO: 156), allowing the translation of two products. BCL-2 family members contain a pattern of BH1-4 sequences that bind and neutralize the ability of pro-apoptotic BH3-containing BIM, BAD, and BID to bind to pore-forming BAX/BAK at the mitochondrial outer membrane. The protein survivin functions by inhibiting caspase activation. The inducible oligonucleotide expression delays EBV targeting until anti-apoptotic protein levels are ramped up. The co-expression construct is a safeguard to ensure that the transactivator is only expressed with anti-apoptotic protein expression.

In another embodiment, a combination of antisense oligonucleotides from SEQ ID NOs: 27-146 is delivered together with BCL-2-member proteins (SEQ ID NOs: 157-162), or and/or survivin protein (SEQ ID NO: 163).

The oligonucleotides function by blocking mRNA translation through RNase-H cleavage, steric hindrance, or by direct RNA-induced silencing complex cleavage. Antisense oligonucleotides are preferred over short interfering RNA because the EBV miRNA-BART6-5p targets Dicer mRNA. The sequences in SEQ ID NOs: 27-146 are derived from a prototypical isolate (LN827555 or AJ507799). Patients can harbor different strains and multiples thereof, altering oligonucleotide target complement sequences. Thus, oligonucleotide target sequences were chosen from conserved regions across isolates.

In some instances, the constructs are DNA plasmids or linearized. In some instances, the gene coding sequences use codon optimization for increased translation efficiency. Online tools are available free for sequence codon optimization (Fuglsang 2003). In some instances, the cDNA contains an artificial or natural intron to increase transcription efficiency (SEQ ID NO: 164).

EBV infection can also be eliminated by targeting EBV genes with TALENS, meganucleases, zinc finger nucleases, or a Clustered Regularly Interspaced Short Palindromic Repeats (CRISPR)/CRISPR-associated nuclease (Cas) system. A designer meganuclease significantly reduced human herpes simplex 1 infection in the superior cervical ganglia and trigeminal ganglia of mice (Aubert et al. 2020; Aubert et al. 2016). Aubert et al. found meganucleases more efficient than CRISPR/Cas9 editing, however other studies show CRISPR/Cas gene editing is more successful (Park et al. 2019).

In vitro CRISPR/Cas editing methods for eliminating EBV infection have been described previously (Wang and Quake 2014; Noh et al. 2016; van Diemen et al. 2016; Yuen et al. 2015). Wang and Quake reported that targeting multiple EBV latency expressed proteins with complementary guide strands increased elimination efficacy. As discussed earlier, EBV genome elimination will cause cell-mediated apoptosis. Therefore, preventing cell-mediated apoptosis in critical cells is necessary for successful EBV infection treatment.

In some embodiments, a CRISPR/Cas gene-editing system is controlled by an inducible reverse tetracycline-transactivator (SEQ ID NO: 147), wherein guide strands target genes encoding one or more EBV proteins. In some instances, targets comprise the flanking ends of EBNA-1 (SEQ ID NOs: 165-166), repeat regions (SEQ ID NOs: 167-168), 5′ exon of EBNA-2 (SEQ ID NOs: 169-170), and 5′ exon of latent membrane protein-1 (SEQ ID NOs: 171-172). In some instances, the guide strands and Cas nuclease (SEQ ID NO: 173) reside on the same construct, with expression under the control of the tetracycline-transactivator transcription domain (SEQ ID NO: 148). In some instances, an inducible-transactivator controls Cas transcription. In some instances, the human U6 promoter (SEQ ID NO: 174) or minimal cytomegalovirus promoter (SEQ ID NO: 175) with enhancer (SEQ ID NO: 176) regulates guide strand transcription. In some instances, a Cas9 variant is used. In some instances, the Cas nuclease contains a nuclear localization signal (SEQ ID NO: 177) at the N- or C-termini. The CRISPR/Cas system is delivered encapsulated together with a construct for co-expression of the reverse tetracycline-transactivator and cDNA for BCL-2 family members BCL-2, BCL-XL, MCL-1, BFL-1, BCL-W, and BCL2L10 (SEQ ID NOs: 149-154) and/or survivin (BIRC5) (SEQ ID NO: 155). In some instances, transcription of the transactivator and anti-apoptotic construct is regulated by sequences comprising the human U6 promoter (SEQ ID NO: 174), proprietary Pmax promoter, minimal cytomegalovirus promoter (SEQ ID NO: 175), and enhancer (SEQ ID NO: 176). In some instances, the constructs are within plasmids. In some instances, the constructs are linearized. In some instances, the cDNA contains an artificial or natural intron to increase transcription efficiency. In some instances, the gene coding sequences use codon optimization.

In some embodiments, the CRISPR/Cas and guide strand construct are delivered with BCL-2 family member protein (SEQ ID NOs: 157-162) and/or survivin protein (SEQ ID NO: 163).

Non-limiting examples of suitable Cas proteins which may be used in accordance with the present teachings include Cas3, Cas4, Cas5, Cas5e (or CasD), Cas6, Cas6e, Cas6f, Cas7, Cas8a1, Cas8a2, Cas8b, Cas8c, Cas9, Cas 10, Cas1 Od, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csxl7, Csxl4, CsxlO, Csxl6, CsaX, Csx3, Cszl, Csxl5, Csf1, Csf2, Csf3, Csf4, and Cul966. Cas9 is a monomeric DNA nuclease guided to a DNA target sequence adjacent to the guide strand's protospacer adjacent motif (PAM). The Cas9 protein comprises two nuclease domains homologous to RuvC and HNH nucleases. The HNH nuclease domain cleaves the complementary DNA strand whereas the RuvC-like domain cleaves the non-complementary strand and, as a result, a blunt cut is introduced in the target DNA. The methods for CRISPR/Cas gene editing are constantly being improved and have moved beyond ex vivo genetic modification. A clinical trial (NCT04601051) is currently testing in vivo CRISP/Cas gene editing delivered in lipid nanoparticles for Hereditary Transthyretin Amyloidosis with Polyneuropathy. U.S. Pat. No. 10,760,075 describes using CRISP/Cas to treat a microbial infection along with immunotherapy treatment. Herein gene expression must be introduced in combination with CRISP/Cas gene editing to prevent cell death of critical cells.

Solid lipids are complexed with the RNA or DNA payload in lipid nanoparticles. Liposomes are unilamellar or multilamellar vesicles that have a lipophilic membrane and an aqueous interior containing the composition to be delivered. While the production and lipid arrangement in nanoparticles and liposomes differ, they are typically made from the same cationic compositions.

Advantages of liposomes include: liposomes obtained from natural phospholipids are biocompatible and biodegradable; liposomes can incorporate a wide range of water and lipid-soluble drugs; liposomes protect encapsulated drugs in their internal compartments from metabolism and degradation (Lieberman et al. 1989). Important considerations in the preparation of liposome formulations are the lipid surface charge, vesicle size, and the aqueous volume of the liposomes.

Liposomes that are pH-sensitive or negatively charged, entrap RNA rather than complex with it. Since the RNA and the lipid are similarly charged, repulsion rather than complex formation occurs. Nevertheless, some RNA is entrapped within the aqueous interior of these liposomes. pH-sensitive liposomes have been used to deliver mRNA encoding the thymidine kinase gene to cell monolayers in culture. Expression of the exogenous gene was detected in the target cells (Zhou and Huang 1992).

Because liposomal membranes are structurally similar to biological membranes, liposomes merge with the cellular membranes and empty contents into the cell. Liposomes fall into two broad classes. Cationic liposomes are positively charged liposomes that interact with the negatively charged DNA molecules to form a stable complex. The positively charged DNA/liposome complex binds to negatively charged cell surface and is internalized in an endosome. Liposomes within endosomes can be enzymatically degraded as the endosome matures and merges with lysosomes. Thus, endosomal digestion is a major barrier for in vivo intracellular drug delivery. To avoid this, the liposome must rupture the endosomal membrane to release contents into the cell. Including cationic or ionizable groups, such as amines, on the liposome surface is one way to accomplish endosomal escape. The protonatable amine group becomes cationic as the pH decreases. Cationic groups neutralize the negative change between the endosomal membrane and liposome destabilizing the membrane and result in liposomal contents released into the cell cytoplasm (Martens et al. 2014).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine such as soybean phosphatidylcholine and egg phosphatidylcholine. Another type is formed from mixtures of phospholipid and/or phosphatidylcholine and/or cholesterol.

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome (A) comprises one or more glycolipids, such as monosialoganglioside or (B) is derivatized with one or more hydrophilic polymers, such as a polyethylene glycol (PEG) moiety. Sterically stabilized liposomes containing gangliosides, sphingomyelin, or PEG-derivatized lipids have enhanced circulation half-life from reduced reticuloendothelial uptake (Allen and Chonn 1987).

Many liposomes comprising lipids derivatized with one or more hydrophilic polymers, and methods of preparation thereof, are known in the art (Moncalvo et al. 2020). Synthetic phospholipids modified by the attachment of carboxylic groups of polyalkylene glycols (e.g., PEG) are described by Sears (U.S. Pat. Nos. 4,426,330 and 4,534,899). Klibanov et al. described experiments demonstrating that liposomes comprising phosphatidylethanolamine (PE) derivatized with PEG or PEG stearate have significant increases in blood circulation half-lives (Klibanov et al. 1990). Blume et al. extended such observations to other PEG-derivatized phospholipids, e.g., DSPE-PEG, formed from the combination of distearoylphosphatidylethanolamine (DSPE) and PEG (Blume and Cevc 1990). Liposomes having covalently bound PEG moieties on their external surface are described in European Patent No. EP 0 445 131 B1 and WO 90/04384. Liposome compositions containing 1-20 mole percent of PE derivatized with PEG, and methods of use thereof, are described in U.S. Pat. No. 5,013,556.

Lipid nanoparticles and liposomes are useful for the delivery of active ingredients to the site of action. Delivering a combination of therapeutics in one package can ensure that each cell receives the necessary compositions in correct proportions. Targeting the liposome delivery to a specific cell surface with targeting moieties can improve delivery. For instance, antibody targeting liposomes increase delivery efficiency (Wang and Huang 1987b; Wang and Huang 1987a). Meissner et al. treated mice with CD20 targeting liposomes comprising BCL-2 antisense oligonucleotide to eliminate the B-cell lymphoma tumors in the mice (Meissner et al. 2015). Increased delivery efficiency increases therapeutic efficacy while minimizing side effects.

U.S. Pat. Nos. 5,540,935 and 5,556,948 describes PEG-containing liposomes derivatized with functional moieties on their surface. In some embodiments, liposomes are conjugated with any of the same functional targeting ligands described above in paragraph [0042] for direct oligonucleotide conjugation. In some instances, endothelial cells expressing glucose transporter-1 are targeted with glucose conjugated liposomes (Min et al. 2020). Techniques for conjugating ligands to the liposome surface, both covalently and non-covalently, are known art. In some instances, the targeting moiety is conjugated to the liposome using an amide bond formation, thiol bond formation, hydrazone bond formation, ester bond formation, or an avidin-biotin bond (Ero\check{g}lu and İbrahim 2020).

WO 96/40062 discloses methods for encapsulating high molecular weight nucleic acids in liposomes. U.S. Pat. No. 5,264,221 discloses protein-bonded liposomes containing dsRNA. U.S. Pat. No. 5,665,710 describes methods of encapsulating oligodeoxynucleotides in liposomes. WO 97/04787 discloses liposomes comprising dsRNAs targeted to the Raf gene. A neutral liposomal formulation containing antisense oligonucleotide complementary to growth factor receptor-bound protein 2 (Grb2) mRNA is currently being tested in clinical trials for multiple cancer types. Patisiran (Onpattro) is a treatment approved for familial amyloid polyneuropathy that delivers double-stranded RNA in a liposome.

In another embodiment, the constructs are delivered in a naked plasmid or polynucleotide. Neovasculgen is a plasmid encoding VEGF for the treatment of peripheral artery disease. Pegaptanib (Macugen) is a polynucleotide encoding VEFG for the treatment of macular degeneration. In another embodiment, the constructs are delivered using a viral vector. Viral vectors are made safe by deleting virulence factors. Viral vectors may be necessary for high transduction efficiency or expression. High expression of BCL-2 family member proteins may be necessary to prevent apoptosis. Gene therapy treatments using adenovirus and lentiviral vectors are the leading platforms. Zolgensma (onasemnogene) is an incompetent recombinant adenovirus 9 that delivers the SMN gene by intravenous injection. The human immunodeficiency type 1 lentivirus vector is used to transform human erythroid cells ex vivo with the β-globin gene for sickle cell treatment (Uchida et al. 2019). The persistence of viral vectors could be risky. However, a modified lentivirus vector can self-inactivate, preventing viral RNA replication (Zufferey et al. 1998).

The described therapeutics can be delivered by intravenous, intradermal, subcutaneous, intramuscular, or intracranial injection. However, therapeutic delivery could temporarily increase a local inflammatory response. To prevent therapy-related thrombosis, an oral thrombin inhibitor, such as Dabigatran can be administered to patients before treatment (Cortes-Canteli et al. 2019). Patients are administered doxycycline by oral route to induce the expression of constructs containing tetracycline-transactivator domains.

### Definitions

The term plus means sense or coding strand. The term minus means antisense or complementary to the coding strand. The term “antiparallel” means in the opposite direction or 3′ to 5′ direction, while forward is the 5′ to 3′ direction or left to right. Herein, the definition of an isolate is a virus derived from an individual, it may or may not constitute a separate strain. A moiety can be a ligand, peptide, oligonucleotide, antibody, antibody fragment, glucose, or any targeting molecule that connects the oligonucleotide to a cell surface biomarker or connects the liposome to a cell surface biomarker. A recombinant virus is defined as a virus that is modified from its original sequence. The abbreviation ncRNA stands for non-coding RNA. Non-coding RNA is RNA that is not processed into mRNA for protein-coding and may be intergenic or intragenic. The term incompetent means that the virus cannot replicate. The term liposome generally means a vesicle composed of amphiphilic lipids arranged in a spherical shape with an aqueous core. The term lipid nanoparticle generally means the payload is complexed with a solid cationic lipid. Micelles and liposomes can coexist in some lipid nanoparticles formulations. An expression construct is a double-stranded DNA nucleotide sequence that includes sequences for transcript replication. An expression construct may be circularized, as in a plasmid, or linear.

### REFERENCES

Abramson, B. L.; Srivaratharajah, K.; Davis, L. L.; Parapid (2018): Women and Hypertension: Beyond the 2017 Guideline for Prevention, Detection, Evaluation, and Management of High Blood Pressure in Adults. Available online at https://www.acc.org/latest-in-cardiology/articles/2018/07/27/09/02/women-and-hypertension.
Allen, T. M.; Chonn, A. (1987): Large unilamellar liposomes with low uptake into the reticuloendothelial system. In FEBS letters 223 (1), pp. 42-46. DOI: 10.1016/0014-5793(87)80506-9.
Astriab-Fisher, Anna; Sergueev, Dimitri. Fisher, Michael; Shaw, Barbara Ramsay; Juliano, R. L. (2002): Conjugates of antisense oligonucleotides with the Tat and antennapedia cell-penetrating peptides. Effects on cellular uptake, binding to target sequences, and biologic actions. In Pharmaceutical research 19 (6), pp. 744-754. DOI: 10.1023/a:1016136328329.
Aubert, Martine; Madden, Emily A.; Loprieno, Michelle; DeSilva Feelixge, Harshana S.; Stensland, Laurence; Huang, Meei-Li et al. (2016): In vivo disruption of latent HSV by designer endonuclease therapy. In JCI insight 1 (14). DOI: 10.1172/jci.insight.88468.
Aubert, Martine; Strongin, Daniel E.; Roychoudhury, Pavitra; Loprieno, Michelle A.; Haick, Anoria K.; Klouser, Lindsay M. et al. (2020): Gene editing and elimination of latent herpes simplex virus in vivo. In Nature communications 11 (1), p. 4148. DOI: 10.1038/s41467-020-17936-5.
Baird, Alison L.; Westwood, Sarah; Lovestone, Simon (2015): Blood-Based Proteomic Biomarkers of Alzheimer's Disease Pathology. In Frontiers in neurology 6, p. 236. DOI: 10.3389/fneur.2015.00236.
Barrio-Alonso, E.; Hernandez-Vivanco, A.; Walton, C. C.; Perea, G.; Frade, J. M. (2018): Cell cycle reentry triggers hyperploidization and synaptic dysfunction followed by delayed cell death in differentiated cortical neurons. In Scientific reports 8 (1), p. 14316. DOI: 10.1038/s41598-018-32708-4.
Barthelson, Karissa Newman, Morgan; Lardelli, Michael (2020): Sorting Out the Role of the Sortilin-Related Receptor 1 in Alzheimer's Disease. In Journal of Alzheimer's disease reports 4 (1), pp. 123-140. DOI: 10.3233/ADR-200177.
Bennett, S. A. L; Pappas, B. A; Stevens, W. D; Davidson. C. M; Fortin. T.; Chen, J. (2000): Cleavage of amyloid precursor protein elicited by chronic cerebral hypoperfusion. In Neurobiology of aging 21 (2). pp. 207-214. DOI: 10.1016/S0197-4580(00)00131-7.
Biswas, Subhas C.; Shi, Yijie; Vonsattel, Jean-Paul G.; Leung, Conrad L.; Troy, Carol M.; Greene, Lloyd A. (2007): Bim is elevated in Alzheimer's disease neurons and is required for beta-amyloid-induced neuronal apoptosis. In The Journal of neuroscience: the official journal of the Society for Neuroscience 27 (4), pp. 893-900. DOI: 10.1523/JNEUROSCI.3524-06.2007.
Blume, Gabriele; Cevc, Gregor (1990): Liposomes for the sustained drug release in vivo. In Biochimica et Biophysica Acta (BBA)—Biomembranes 1029 (1), pp. 91-97. DOI: 10.1016/0005-2736(90)90440-Y.
Braak, H.; Braak, E. (1991): Neuropathological staging of Alzheimer-related changes. In Acta Neuropathol 82 (4). pp. 239-259. DOI: 10.1007/BF00308809.
Brenowitz, Willa D.; Nelson, Peter T.; Besser, Lilah M.; Heller, Katherine B.; Kukull, Walter A. (2015): Cerebral amyloid angiopathy and its co-occurrence with Alzheimer's disease and other cerebrovascular neuropathologic changes. In Neurobiology of aging 36 (10). pp. 2702-2708. DOI: 10.1016/j.neurobiolaging.2015.06.028.
Calderwood, Michael A.; Venkatesan, Kavitha; Xing, Li; Chase, Michael R.; Vazquez, Alexei; Holthaus, Amy M. et al. (2007): Epstein-Barr virus and virus human protein interaction maps. In Proceedings of the National Academy of Sciences 104 (18), pp. 7606-7611. DOI: 10.1073/pnas.0702332104.
Casiraghi, Costanza; Dorovini-Zis, Katerina; Honitz, Marc S. (2011): Epstein-Barr virus infection of human brain microvessel endothelial cells. A novel role in multiple sclerosis. In Journal of neuroimmunology 230 (1-2), pp. 173-177. DOI: 10.1016/j.jneuroim.2010.08.003.
Chai, Qingqing; Jovasevic, Vladimir, Malikov, Viacheslav; Sabo, Yosef; Morham, Scott; Walsh, Derek; Naghavi, Mojgan H. (2017): HIV-1 counteracts an innate restriction by amyloid precursor protein resulting in neurodegeneration. In Nature communications 8 (1), p. 1522. DOI: 10.1038/s41467-017-01795-8.
Chandra, Avinash (2015): Role of amyloid from a multiple sclerosis perspective. A literature review. In Neuroimmunomodulation 22 (6), pp. 343-346. DOI: 10.1159/000375309.
Chen, Huang-Hsi; Pemg, Wuu-Tsun; Chiou, Jeng-Yuan; Wang, Yu-Hsun; Huang, Jing-Yang; Wei, James Cheng-Chung (2019): Risk of dementia among patients with Sjogren's syndrome. A nationwide population-based cohort study in Taiwan. In Seminars in arthritis and rheumatism 48 (5). pp. 895-899. DOI: 10.1016/j.semarthrit.2018.06.007.
Chen, Yunjia; Peng, Yin; Che, Pulin; Gannon, Mary; Liu, Yin; Li, Ling et al. (2014): α(2A) adrenergic receptor promotes amyloidogenesis through disrupting APP-SorLA interaction. In Proceedings of the National Academy of Sciences of the United States of America 111 (48), pp. 17296-17301. DOI: 10.1073/pnas.1409513111.
Cheng, Shiyue; Li, Zhi; He, Junju; Fu, Shujun; Duan, Yumei; Zhou, Qin et al. (2019): Epstein-Barr virus noncoding RNAs from the extracellular vesicles of nasopharyngeal carcinoma (NPC) cells promote angiogenesis via TLR3/RIG-I-mediated VCAM-1 expression. In Biochimica et biophysica acta. Molecular basis of disease 1865 (6), pp. 1201-1213. DOI: 10.1016/j.bbadis.2019.01.015.
Chung, Sun Ju; Kim, Mi-Jung; Kim, Young Jin Kim, Juyeon; You, Sooyeoun; Jang, Eun Hye et al. (2014): CR1, ABCA7, and APOE genes affect the features of cognitive impairment in Alzheimer's disease. In Journal of the neurological sciences 339 (1-2), pp. 91-96. DOI: 10.1016/j.jns.2014.01.029.
Ciaramella, Antonio; Salani, Francesca; Bizzoni, Federica; Orfei, Maria Donata; Caltagirone, Carlo; Spalletta, Gianfranco; Bossu, Paola (2016): Myeloid dendritic cells are decreased in peripheral blood of Alzheimer's disease patients in association with disease progression and severity of depressive symptoms. In J Neuroinflammation 13 (1), p. 383. DOI: 10.1186/s12974-016-0483-0.
Ciarlo, Eleonora; Massone, Sara; Penna, Ilaria Nizzari, Mario; Gigoni, Arianna; Dieci, Giorgio et al. (2013): An intronic ncRNA-dependent regulation of SORL1 expression affecting Aβ formation is upregulated in post-mortem Alzheimer's disease brain samples. In Disease models & mechanisms 6 (2), pp. 424-433. DOI: 10.1242/dmm.009761.
Collard, C. D.; Bukusoglu, C.; Agah, A.; Colgan, S. P.; Reenstra, W. R.; Morgan, B. P.; Stahl, G. L (1999): Hypoxia-induced expression of complement receptor type 1 (CR1, CD35) in human vascular endothelial cells. In The American journal of physiology 276 (2), C450-8. DOI: 10.1152/ajpcell.1999.276.2.C450.
Cortes-Canteli, Marta; Kruyer, Anna; Femandez-Nueda, Irene; Marcos-Diaz, Ana; Ceron, Carlos; Richards, Allison T. et al. (2019): Long-Term Dabigatran Treatment Delays Alzheimer's Disease Pathogenesis in the TgCRND8 Mouse Model. In Journal of the American College of Cardiology 74 (15), pp. 1910-1923. DOI: 10.1016/j.jacc.2019.07.081.
Croia, Cristina; Astorri, Elisa; Murray-Brown, William; Willis, Amanda; Brokstad, Karl A.; Sutcliffe, Nurhan et al. (2014): Implication of Epstein-Barr virus infection in disease-specific autoreactive B cell activation in ectopic lymphoid structures of Sjögren's syndrome. In Arthritis & rheumatology (Hoboken, N.J.) 66 (9), pp. 2545-2557. DOI: 10.1002/art.38726.
D′Anna, Lucio; Abu-Rumeileh, Samir; Fabris, Martina; Pistis, Cinzia; Baldi, Antonio; Sanvilli, Nova et al. (2017): Serum Interleukin-10 Levels Correlate with Cerebrospinal Fluid Amyloid Beta Deposition in Alzheimer Disease Patients. In Neuro-degenerative diseases 17 (4-5), pp. 227-234. DOI: 10.1159/(00474940.
Desai, Brinda S.; Schneider, Julie A.; Li, Jia-Liang; Carvey, Paul M.; Hendey, Bill (2009): Evidence of angiogenic vessels in Alzheimer's disease. In J. of Neural Transmission 116 (5), pp. 587-597. DOI: 10,1007/s00702-009-0226-9.
Di Carlo, Paola; Trizzino, Marcello; Titone, Lucina; Capra, Giuseppina; Colletti, Piero; Mazzola, Giovanni et al. (2011): Unusual MRI findings in an immunocompetent patient with EBV encephalitis. A case report. In BMC Med Imaging 11 (1), p. 481. DOI: 10.1186/1471-2342-11-6.
Eimer, William A.; Vijaya Kumar, Deepak Kumar; Navalpur Shanmugam, Nanda Kumar; Rodriguez, Alex S.; Mitchell, Teryn; Washicosky, Kevin J. et al. (2018): Alzheimer's Disease-Associated β-Amyloid Is Rapidly Seeded by Herpesviridae to Protect against Brain Infection. In Neuron 99 (1), 56-63.e3. DOI: 10.1016/j.neuron.2018.06.030.
Ero\check{g}lu, İpek; İbrahim, Mamudu (2020): Liposome-ligand conjugates. A review on the current state of art. In Journal of drug targeting 28 (3), pp. 225-244. DOI: 10.1080/1061186X.2019.1648479.
Farrell, Christopher J.; Lee, Jae Myun; Shin, Eui-Cheol; Cebrat, Marek; Cole, Philip A.; Hayward, S. Diane (2004): Inhibition of Epstein-Barr virus-induced growth proliferation by a nuclear antigen EBNA2-TAT peptide. In Proceedings of the National Academy of Sciences 101 (13), pp. 4625-4630. DOI: 10.1073/pnas.0306482101.
Fonseca, Maria I.; Chu, Shuhui; Pierce, Aimee L.; Brubaker, William D.; Hauhart, Richard E.; Mastroeni, Diego et al. (2016): Analysis of the Putative Role of CR1 in Alzheimer's Disease. Genetic Association, Expression and Function. In PloS one 11 (2), e0149792. DOI: 10.1371/joumal.pone.0149792.
Frade, José M.; López-Sánchez, Noelia (2017): Neuronal tetraploidy in Alzheimer and aging. In Aging 9 (10), pp. 2014-2015. DOI: 10.18632/aging.101312.
Frenkel-Denkberg, Galit; Gershon, David; Levy, Andrew P. (1999): The function of hypoxia-inducible factor 1 (HIF-1) is impaired in senescent mice. In FEBS letters 462 (3), pp. 341-344. DOI: 10.1016/S0014-5793(99))01552-5.
Fuglsang, Anders (2003): Codon optimizer. A freeware tool for codon optimization. In Protein Expression and Purification 31 (2), pp. 247-249. DOI: 10.1016/s1046-5928(03)00213-4.
Fuhrer, Tessa E.; Palpagama, Thulani H.; Waldvogel, Henry J.; Synek, Beth J. L.; Turner, Clinton; Faull, Richard L.; Kwakowsky. Andrea (2017): Impaired expression of GABA transporters in the human Alzheimer's disease hippocampus, subiculum, entorhinal cortex and superior temporal gyrus. In Neuroscience 351, pp. 108-118. DOI: 10.1016/j.neuroscience.2017.03.041.
Gaidin, Sergei G.; Zinchenko, Valery P.; Sergeev, Alexander I.; Teplov, Ilia Y.; Mal'tseva, Valentina N.; Kosenkov, Artem M. (2020): Activation of alpha-2 adrenergic receptors stimulates GABA release by astrocytes. In Gila 68 (6). pp. 1114-1130. DOI: 10.1002/glia.23763.
Gannon, Mary; Che, Pulin; Chen, Yunjia; Jiao, Kai; Roberson, Erik D.; Wang, Qin (2015): Noradrenergic dysfunction in Alzheimer's disease. In Frontiers in neuroscience 9, p. 220. DOI: 10.3389/fnins.2015.00220.
Gate, David; Saligrama, Naresha; Leventhal, Olivia; Yang, Andrew C.; Unger, Michael S.; Middeldorp, Jinte et al. (2020): Clonally expanded CD8 T cells patrol the cerebrospinal fluid in Alzheimer's disease. In Nature 577 (7790), pp. 399-404. DOI: 10.1038/s41586-019-1895-7.
Grammas, Paula; Sanchez, Alma; Tripathy, Debjani; Luo, Ester; Martinez, Joseph (2011): Vascular signaling abnormalities in Alzheimer disease. In Cleveland Clinic journal of medicine 78 Suppl 1, S50-3. DOI: 10.3949/ccjm.78.sl.09.
Greene, Lloyd A.; Liu, David X.; Troy, Carol M.; Biswas. Subhas C. (2007): Cell cycle molecules define a pathway required for neuron death in development and disease. In Biochimica et biophysica acta 1772 (4), pp. 392-401. DOI: 10.1016/j.bbadis.2006.12.003.
Harley, John B., Chen, Xiaoting; Pujato, Mario; Miller, Daniel; Maddox, Avery; Forney, Carmy et al. (2018): Transcription factors operate across disease loci, with EBNA2 implicated in autoimmunity. In Nature genetics 50 (5), pp. 699-707. DOI: 10.1038/s41588-018-0102-3.
Hategan, Alina; Masliah, Eliezer; Nath, Avindra (2019): HIV and Alzheimer's disease. Complex interactions of HIV-Tat with amyloid 1 peptide and Tau protein. In Journal of neurovirology 25 (5). pp. 648-660. DOI: 10.1007/s13365-019-00736-z.
Herrup, Karl (2010): The involvement of cell cycle events in the pathogenesis of Alzheimers disease. In Alzheimer's research & therapy 2 (3), p. 13. DOI: 10.1186/alzrt37.
Holzerlandt, Ria; Orengo, Christine; Kellam, Paul; Alb, M. March (2002): Identification of New Herpesvirus Gene Homologs in the Human Genome. In Genome research 12 (11). pp. 1739-1748. DOI: 10.1101/gr.334302.
Hou, Tsung-Yun; Hsu, Hui-Ching; Lin, Tzu-Min; Chang, Yu-Sheng; Chen, Wei-Sheng; Kuo. Pei-I et al. (2019): Higher risk of dementia in primary Sjogren's syndrome. In Annals of clinical and translational neurology 6 (4), pp. 633-641. DOI: 10.1002/acn3.737.
Iwakiri, Dai; Zhou, Li; Samanta, Mrinal; Matsumoto, Misako; Ebihara, Takashi; Seya, Tsukasa et al. (2009): Epstein-Barr virus (EBV)-encoded small RNA is released from EBV-infected cells and activates signaling from Toll-like receptor 3. In The Journal of experimental medicine 206 (10), pp. 2091-2099. DOI: 10.1084/jem.20081761.
Jabs, W. J.; Paulsen, M.; Wagner, H. J.; Kirchner. H.; Kliter, H. (1999): Analysis of Epstein-Bar virus (EBV) receptor CD21 on peripheral B lymphocytes of long-term EBV-adults. In Clinical & Experimental Immunology 116 (3), pp. 468-473. DOI: 10.1046/j.1365-2249.1999.00912.x.
James, Lisa M.; Christova, Peka; Lewis, Scott M.; Engdahl, Brian E.; Georgopoulos, Angeliki; Georgopoulos, Apostolos P. (2018): Protective Effect of Human Leukocyte Antigen (HLA) Allele DRB1*13:02 on Age-Related Brain Gray Matter Volume Reduction in Healthy Women. In EBioMedicine 29, pp. 31-37. DOI: 10.1016/j.ebiom.2018.02.005.
Jha, Hem Chandra; Mehta, Devan; Lu, Jie; El-Naccache, Darine; Shukla, Sanket K.; Kovacsics, Colleen et al. (2015): Gammaherpesvirus Infection of Human Neuronal Cells. In mBio 6 (6), e01844-15. DOI: 10.1128/mBio.01844-15.
Jog, Neelakshi R.; Chakravarty, Eliza F.; Guthridge. Joel M.; James. Judith A. (2018): Epstein Barr Virus Interleukin 10 Suppresses Anti-inflammatory Phenotype in Human Monocytes. In Frontiers in immunology 9, p. 2198. DOI: 10.3389/fimmu.2018.02198.
Jones, K.; Rivera, C.; Sgadari, C.; Franklin, J.; Max. E. E.; Bhatia, K.; Tosato, G. (1995): Infection of human endothelial cells with Epstein-Barr virus. In The Journal of experimental medicine 182 (5), pp. 1213-1221. DOI: 10.1084/jem.182.5.1213.
Khan, Irfan Sagir; Loh, Kwok Seng; Petersson, Fredrik (2019): Amyloid and hyaline globules in undifferentiated nasopharyngeal carcinoma. In Annals of diagnostic pathology 40, pp. 1-6. DOI: 10.1016/j.anndiagpath.2019.02.016.
Kirino, T.; Tamura, A.; Sano, K. (1984): Delayed neuronal death in the rat hippocampus following transient forebrain ischemia. In Acta Neuropathol 64 (2), pp. 139-147. DOI: 10.1007/BF00695577.
Klibanov, Alexander L.; Maruyama, Kazuo; Torchilin, Vladimir P.; Huang, Leaf (1990): Amphipathic polyethyleneglycols effectively prolong the circulation time of liposomes. In FEBS letters 268 (1), pp. 235-237. DOI: 10.1016/0014-5793(90)81016-h.
Körbelin, Jakob; Dogbevia, Godwin; Michelfelder, Stefan; Ridder, Dirk A.; Hunger, Agnes; Wenzel, Jan et al. (2016): A brain microvasculature endothelial cell-specific viral vector with the potential to treat neurovascular and neurological diseases. In EMBO molecular medicine 8 (6), pp. 609-625. DOI: 10.15252/emmm.201506078.
Korte, Nils; Nortley, Ross; Attwell, David (2020): Cerebral blood flow decrease as an early pathological mechanism in Alzheimer's disease. In Acta Neuropathol 11 (Suppl 1), p. 149. DOI: 10.1007/s00401-020-02215-w.
Kosenko, Elena A.; Tikhonova, Lyudmila A.; Montoliu, Carmina; Barreto, George E.; Aliev, Gjumrakch; Kaminsky, Yury G. (2017): Metabolic Abnormalities of Erythrocytes as a Risk Factor for Alzheimer's Disease. In Frontiers in neuroscience 11, p. 728. DOI: 10.3389/fnins.2017.00728.
Kumar, Deepak Kumar Vijaya; Choi, Se Hoon; Washicosky, Kevin J.; Eimer, William A.; Tucker, Stephanie; Ghofrani, Jessica et al. (2016): Amyloid-β peptide protects against microbial infection in mouse and worm models of Alzheimers disease. In Science translational medicine 8 (340), 340ra72. DOI: 10.1126/scitranslmed.aafl059.
Kurt, M. A.; Davies, D. C.; Kidd, M. (1999): beta-Amyloid immunoreactivity in astrocytes in Alzheimer's disease brain biopsies. An electron microscope study. In Experimental neurology 158 (1), pp. 221-228. DOI: 10.1006/exnr.1999.7096.
Lai, Catherine Jessica; Tay, Boon Hunt (2016): Pharmacophore-based screening targeted at upregulated FN1, MMP-9, APP reveals therapeutic compounds for nasopharyngeal carcinoma. In Computers in biology and medicine 69, pp. 158-165. DOI: 10.1016/j.compbiomed.2015.12.015.
Li, Shaomin; Hong, Soyon; Shepardson, Nina E.; Walsh, Dominic M., Shankar, Ganesh M.; Selkoe, Dennis (2009): Soluble oligomers of amyloid Beta protein facilitate hippocampal long-term depression by disrupting neuronal glutamate uptake. In Neuron 62 (6), pp. 788-801. DOI: 10.1016/j.neuron.2009.05.012.
Lieberman. H. A.; Lachman. L.; Schwartz J. B. (Eds.) (1989): Pharmaceutical dosage forms: Tablets. 2 volumes. New York: Marcel Dekker (1).
Lin, Shih-Yi; Hsu, Wu-Huei; Lin, Cheng-Chieh; Lin, Cheng-Li; Yeh, Hung-Chieh; Kao, Chia-Hung (2019): Association of Transfusion With Risks of Dementia or Alzheimers Disease. A Population-Based Cohort Study. In Frontiers in psychiatry 10, p. 571. DOI: 10.3389/fpsyt.2019.00571.
Liu, Wenhua; Yuen, Eunice Y.; Allen, Patrick B.; Feng, Jian; Greengard, Paul; Yan, Zhen (2006): Adrenergic modulation of NMDA receptors in prefrontal cortex is differentially regulated by RGS proteins and spinophilin. In Proc. Natl. Acad Sci. USA 103 (48), pp. 18338-18343. DOI: 10.1073/pnas.0604560103.
Lu, Rui-Chun; Yang, Wu; Tan, Lin; Sun. Fu-Rong; Tan, Meng-Shan; Zhang, Wei et al. (2017): Association of HLA-DRB1 polymorphism with Alzheimer's disease. A replication and meta-analysis. In Oncotarget 8 (54), pp. 93219-93226. DOI: 10.18632/oncotarget.21479.
Luczynski, Pauline; Laule, Comelia; Hsiung, Ging-Yuek Robin; Moore, G. R. Wayne; Tremlett, Helen (2019): Coexistence of Multiple Sclerosis and Alzheimer's disease. A review. In Multiple sclerosis and related disorders 27, pp. 232-238. DOI: 10.1016/j.msard.2018.10.109.
Martens, Thomas F.; Remaut, Katrien; Demeester, Jo; Smedt, Stefaan C. de; Braeckmans, Kevin (2014): Intracellular delivery of nanomaterials. How to catch endosomal escape in the act. In Nano Today 9 (3), pp. 344-364. DOI: 10.1016/j.nantod.2014.04.011.
McCarthy, Jeanette J.; Saith, Sunita; Linnertz, Colton; Burke, James R.; Hulette, Christine M.; Welsh-Bohmer, Kathleen A.; Chiba-Falek, Omit (2012): The Alzheimer's associated 5′ region of the SORL1 gene cis regulates SORL 1 transcripts expression. In Neurobiology of aging 33 (7), 1485.e1-8. DOI: 10.1016/j.neurobiolaging.2010.10.004.
Mechelli, Rosella; Manzari, Caterina; Policano, Claudia; Annese, Anita; Picardi, Emesto; Umeton, Renato et al. (2015): Epstein-Barr virus genetic variants are associated with multiple sclerosis. In Neurology 84 (13), pp. 1362-1368. DOI: 10.1212/WNL.0000000000001420.
Meissner, Justyna M.; Toporkiewicz, Monika; Czogalla, Aleksander; Matusewicz, Lucyna; Kuliczkowski, Kazimierz; Sikorski, Aleksander F. (2015): Novel antisense therapeutics delivery systems. In vitro and in vivo studies of liposomes targeted with anti-CD20 antibody. In Journal of controlled release: official journal of the Controlled Release Society 220 (Pt A), pp. 515-528. DOI: 10.1016/j.jconrel.2015.11.015.
Min, Hyun Su; Kim, Hyun Jin Naito, Mitsuru Ogura, Satomi; Toh, Kazuko; Hayashi, Kotaro et al. (2020): Systemic Brain Delivery of Antisense Oligonucleotides across the Blood-Brain Barrier with a Glucose-Coated Polymeric Nanocarrier. In Angewandte Chemie (International ed. in English) 59 (21), pp. 8173-8180. DOI: 10.1002/anie.201914751.
Miyakawa, T. (1997): Electron microscopy of amyloid fibrils and microvessels. In Annals of the New York Academy of Sciences 826, pp. 25-34. DOI: 10.1111/j.1749-6632.1997.tb48458.x.
Moncalvo, Filippo; Martinez Espinoza, Maria Isabel; Cellesi, Francesco (2020): Nanosized Delivery Systems for Therapeutic Proteins. Clinically Validated Technologies and Advanced Development Strategies. In Frontiers in bioengineering and biotechnology 8, p. 89. DOI: 10.3389/fbioe.2020.00089.
Nassif, Samer; Ozdemirli, Metin (2013): EBV-positive low-grade marginal zone lymphoma in the breast with massive amyloid deposition arising in a heart transplant patient. A report of an unusual case. In Pediatric transplantation 17 (6), E141-5. DOI: 10.1111/petr.12111.
Ni, Yanxiang; Zhao, Xiaohui; Bao, Guobin; Zou, Lin; Teng, Lin; Wang, Zhu et al. (2006): Activation of beta2-adrenergic receptor stimulates gamma-secretase activity and accelerates amyloid plaque formation. In Nature medicine 12 (12), pp. 1390-1396. DOI: 10.1038/nml485.
Noh, Ka-Won; Park, Jihvun; Kang, Myung-Soo (2016): Targeted disruption of EBNA1 in EBV-infected cells attenuated cell growth. In BMB Reports 49 (4), pp. 226-231. DOI: 10.5483/BMBRep.2016.49.4.260.
Nortley, Ross; Korte, Nils; Izquierdo, Pablo; Hirunpattarasilp, Chanawee; Mishra, Anusha; Jaunmuktane, Zane et al. (2019): Amyloid D oligomers constrict human capillaries in Alzheimer's disease via signaling to pericytes. In Science (New York, N.Y.) 365 (6450). DOI: 10.1126/science.aav9518.
Ogembo, Javier G.; Kannan, Lakshmi; Ghiran, Ionita; Nicholson-Weller, Anne; Finberg, Robert W.; Tsokos, George C.; Fingeroth, Joyce D. (2013): Human complement receptor type 1/CD35 is an Epstein-Barr Virus receptor. In Cell reports 3 (2), pp. 371-385. DOI: 10.1016/j.celrep.2013.01.023.
Ounanian, Annette; Guilbert, Brigitte; Seigneurin, Jean-Marie (1992): Characteristics of Epstein-Barr virus transformed B cell lines from patients with Alzheimer's disease and age-matched controls. In Mechanisms of ageing and development 63 (1), pp. 105-116. DOI: 10.1016/0047-6374(92)90020-e.
Panikkar, Archana; Smith, Corey; Hislop, Andrew; Tellam, Nick; Dasari, Vijayendra; Hogquist, Kristin A. et al. (2015): Cytokine-Mediated Loss of Blood Dendritic Cells During Epstein-Barr Virus-Associated Acute Infectious Mononucleosis. Implication for Immune Dysregulation. In The Journal of infectious diseases 212 (12), pp. 1957-1961. DOI: 10.1093/infdis/jiv340.
Park, Hanseul; Oh. Jungju; Shim. Gayong; Cho, Byounggook; Chang, Yujung; Kim, Siyoung et al. (2019): In vivo neuronal gene editing via CRISPR-Cas9 amphiphilic nanocomplexes alleviates deficits in mouse models of Alzheimer's disease. In Nature neuroscience 22 (4), pp. 524-528. DOI: 10.1038/s41593-019-0352-0.
Parks, Christine G.; Cooper, Glinda S.; Hudson, Lori L.; Dooley, Mary Anne; Treadwell, Edward L.; St Clair, E. W. et al. (2005): Association of Epstein-Barr virus with systemic lupus erythematosus. Effect modification by race, age, and cytotoxic T lymphocyte-associated antigen 4 genotype. In Arthritis and rheumatism 52 (4), pp. 1148-1159. DOI: 10.1002/art.20997.
Reijmer, Yael D.; van Veluw, Susanne J.; Greenberg, Steven M. (2016): Ischemic brain injury in cerebral amyloid angiopathy. In Journal of cerebral blood flow and metabolism: official journal of the International Society of Cerebral Blood Flow and Metabolism 36 (1). pp. 40-54. DOI: 10.1038/jcbfm.2015.88.
Reynolds, Chandra A.; Zavala, Catalina; Gatz, Margaret; Vie, Loryana; Johansson, Boo; Malmberg, Bo et al. (2013): Sortilin receptor 1 predicts longitudinal cognitive change. In Neurobiology of aging 34 (6), 1710.e11-8. DOI: 10.1016/j.neurobiolaging.2012.12.006.
Rivera-Soto, Ricardo; Damania, Blossom (2019): Modulation of Angiogenic Processes by the Human Gammaherpesviruses, Epstein-Barr Virus and Kaposi's Sarcoma-Associated Herpesvirus. In Frontiers in microbiology 10, p. 1544. DOI: 10.3389/fmicb.2019.01544.
Satou, Ryousuke; Penrose, Harrison; Navar, L. Gabriel (2018): Inflammation as a Regulator of the Renin-Angiotensin System and Blood Pressure. In Current hypertension reports 20 (12), p. 100. DOI: 10.1007/s11906-018-0900-0.
Sheng, Morgan; Ertürk, Ali (2014): Long-term depression. A cell biological view. In Philosophical transactions of the Royal Society of London. Series B. Biological sciences 369 (1633), p. 20130138. DOI: 10.1098/rstb.2013.0138.
Simeoni, Federica; Morns, May C.; Heitz, Frederic; Divita, Gilles (2003): Insight into the mechanism of the peptide-based gene delivery system MPG. Implications for delivery of siRNA into mammalian cells. In Nucleic acids research 31 (11), pp. 2717-2724. DOI: 10.1093/nar/gkg385.
Tabata, Takako, Kawakatsu, Hisaaki; Maidji, Ekaterina; Sakai, Takao; Sakai, Keiko; Fang-Hoover, June et al. (2008): Induction of an epithelial integrin alphavbeta6 in human cytomegalovirus-infected endothelial cells leads to activation of transforming growth factor-beta1 and increased collagen production. In The American Journal of Pathology 172 (4), pp. 1127-1140. DOI: 10.2353/ajpath.2008.070448.
Tang, L-Q; Chen, Q-Y; Guo, S-S; Chen, W-H; Li, C-F; Zhang, L. et al (2014): The impact of plasma Epstein-Barr virus DNA and fibrinogen on nasopharyngeal carcinoma prognosis. An observational study. In British journal of cancer 111 (6), pp. 1102-1111. DOI: 10.1038/bjc.2014.393.
Tian, Tian; Zhang, Hui-Xin; He, Chun-Peng; Fan, Song; Zhu, Yan-Liang; Qi, Cui et al. (2018): Surface functionalized exosomes as targeted drug delivery vehicles for cerebral ischemia therapy. In Biomaterials 150, pp. 137-149. DOI: 10.1016/j.biomaterials.2017.10.012.
Timens, W.; Boes, A.; Vos, H.; Poppema, S. (1991): Tissue distribution of the C3d/EBV-receptor. CD21 monoclonal antibodies reactive with a variety of epithelial cells, medullary thymocytes, and peripheral T-cells. In Histochemistry 95 (6), pp. 605-611. DOI: 10.1007/BF00266748.
Toczyski, D. P.; Matera, A. G.; Ward, D. C.; Steitz, J. A. (1994): The Epstein-Barr virus (EBV) small RNA EBER1 binds and relocalizes ribosomal protein L22 in EBV-infected human B lymphocytes. In Proc. Natl. Acad Sci. U.S.A 91 (8), pp. 3463-3467. DOI: 10.1073/pnas.91.8.3463.
Toyama, Kensuke; Spin, Joshua M.; Deng, Alicia C.; Huang, Ting-Ting; Wei, Ke; Wagenhäuser, Markus U. et al. (2018): MicroRNA-Mediated Therapy Modulating Blood-Brain Barrier Disruption Improves Vascular Cognitive Impairment. In Arterioscler Thromb Vasc Biol. 38 (6), pp. 1392-1406. DOI: 10.1161/ATVBAHA. 118.310822.
Tsartsalis, Stergios; Xekardaki, Aikaterini; Hof, Patrick R.; Kövari, Enikö; Bouras, Constantin (2018): Early Alzheimer-type lesions in cognitively normal subjects. In Neurobiology of aging 62, pp. 34-44. DOI: 10.1016/j.neurobiolaging.2017.10.002.
Uchida, Naoya; Hsieh. Matthew M.; Raines, Lydia; Haro-Mora, Juan J.; Demirci, Selami; Bonifacino, Aylin C. et al. (2019): Development of a forward-oriented therapeutic lentiviral vector for hemoglobin disorders. In Nature communications 10 (1), p. 4479. DOI: 10.1038/s41467-019-12456-3.
van Diemen, Ferdy R.; Kruse, Elisabeth M.; Hooykaas, Marjolein J. G.; Bruggeling, Carlijn E.; Schurch, Anita C.; van Ham, Petra M. et al (2016): CRISPR/Cas9-Mediated Genome Editing of Herpesviruses Limits Productive and Latent Infections. In PLoS pathogens 12 (6), e1005701. DOI: 10.1371/joumal.ppat.1005701.
van Oijen, Marieke; Witteman, Jacqueline C.; Hofman, Albert; Koudstaal, Peter J.; Breteler, Monique M. B. (2005): Fibrinogen is associated with an increased risk of Alzheimer disease and vascular dementia. In Stroke 36 (12). pp. 2637-2641. DOI: 10.1161/01.STR.0000189721.31432.26.
Wang, C. Y.; Huang, L. (1987a): pH-sensitive immunoliposomes mediate target-cell-specific delivery and controlled expression of a foreign gene in mouse. In Proceedings of the National Academy of Sciences 84 (22), pp. 7851-7855. DOI: 10.1073/pnas.84.22.7851.
Wang, Chen-Yen; Huang. Leaf (1987b): Plasmid DNA adsorbed to pH-sensitive liposomes efficiently transforms the target cells. In Biochemical and biophysical research communications 147 (3), pp. 980-985. DOI: 10.1016/S0006-291X(87)80166-3.
Wang, Jianbin; Quake, Stephen R. (2014): RNA-guided endonuclease provides a therapeutic strategy to cure latent herpesviridae infection. In Proceedings of the National Academy of Sciences of the United States of America 111 (36), pp. 13157-13162. DOI: 10.1073/pnas.1410785111.
Weeks, J. K.; Helton, K. J.; Conley. M. E.; Onciu, M.; Khan, R. B. (2006): Diffuse CNS vasculopathy with chronic Epstein-Barr virus infection in X-linked lymphoproliferative disease. In AJNR. American journal of neuroradiology 27 (4), pp. 884-886.
Wiener, Julius; Kokotek, Daniel; Rosowski, Simon; Lickert, Heiko; Meier, Matthias (2020): Preparation of single- and double-oligonucleotide antibody conjugates and their application for protein analytics. In Scientific reports 10 (1), p. 1457. DOI: 10.1038/s41598-020-58238-6.
Yajima, Misako; Kanda, Teru; Takada. Kenzo (2005): Critical role of Epstein-Barr Virus (EBV)-encoded RNA in efficient EBV-induced B-lymphocyte growth transformation. In Journal of virology 79 (7), pp. 4298-4307. DOI: 10.1128/JVI.79.7.4298-4307.2005.
Yang, Yan; Mufson, Elliott J.; Herrup, Karl (2003): Neuronal Cell Death Is Preceded by Cell Cycle Events at All Stages of Alzheimer's Disease. In J. Neurosci. 23 (7), pp. 2557-2563. DOI: 10.1523/JNEUROSCI.23-07-02557.2003.
Yin, Huali; Qu, Jiani; Peng, Qiu; Gan, Runliang (2019): Molecular mechanisms of EBV-driven cell cycle progression and oncogenesis. In Medical microbiology and immunology 208 (5), pp. 573-583. DOI: 10.1007/s00430-018-0570-1.
Yuen, Kit-San; Chan, Chi-Ping; Wong, Nok-Hei Mickey; Ho, Chau-Ha; Ho, Ting-Hin; Lei, Ting et al. (2015): CRISPR/Cas9-mediated genome editing of Epstein-Barr virus in human cells. In The Journal of general virology 96 (Pt 3), pp. 626-636. DOI: 10.1099/jgv.0.000012.
Zamolodchikov, D.; Renné, T.; Strickland, S. (2016): The Alzheimer's disease peptide β-amyloid promotes thrombin generation through activation of coagulation factor XII. In Journal of thrombosis and haemostasis:JTH 14 (5), pp. 995-1007. DOI: 10.1111/jth.13209.
Zhang, Fang; Gannon, Mary; Chen, Yunjia; Yan, Shun; Zhang, Sixue; Feng, Wendy et al. (2020): β-amyloid redirects norepinephrine signaling to activate the pathogenic GSK3β/tau cascade. In Science translational medicine 12 (526). DOI: 10.1126/scitranslmed.aay6931.
Zhang, Fang; Gannon, Mary; Chen, Yunjia; Zhou, Lufang; Jiao, Kai; Wang, Qin (2017): The amyloid precursor protein modulates α2A-adrenergic receptor endocytosis and signaling through disrupting arrestin 3 recruitment. In FASEB journal: official publication of the Federation of American Societies for Experimental Biology 31 (10). pp. 4434-4446. DOI: 10.1096/fj.201700346R.
Zhao, Zhuoxian; Rocha, Natalia P.; Salem, Haitham; Diniz, Breno S.; Teixeira, Antonio L. (2018): The association between systemic lupus erythematosus and dementia A meta-analysis. In Dementia & neuropsychologia 12 (2), pp. 143-151. DOI: 10.1590/1980-57642018dn12-020006.
Zhou, Xiaohuai; Huang, Leaf (1992): Targeted delivery of DNA by liposomes and polymers. In Journal of Controlled Release 19 (1-3), pp. 269-274. DOI: 10.1016/0168-3659(92)90082-3.
Zufferey, R. Dull, T.; Mandel, R. J.; Bukovsky, A.; Quiroz, D.; Naldini, L.; Trono, D. (1998): Self-inactivating lentivirus vector for safe and efficient in vivo gene delivery. In J Virol 72 (12), pp. 9873-9880. DOI: 10.1128/JVI.72.12.9873-9880.1998.

1-20. (canceled)

21. An RNA or DNA oligonucleotide drug composed of at least two oligonucleotides, wherein the oligonucleotide sequence is comprised of at least 7 contiguous nucleotides that are at least 70% identical to any of the sequences selected from SEQ ID NOs 3-24, and SEQ ID NOs 27-146.

22. The RNA or DNA oligonucleotide drug according to claim 21, wherein oligonucleotide modifications are comprised of locked nucleic acids, 2′-O-methyl, 2′-O-methoxyethyl, 2′-fluorine, 2′-fluoroarabinonucleic acid, and wherein nucleotide linkages are comprised of phosphorothioate or an amide.

23. Oligonucleotide drug according to claim 22, wherein the selected oligonucleotides are conjugated to a targeting moiety.

24. An oligonucleotide drug according to claim 22, wherein the selected oligonucleotides are complexed in a lipid nanoparticle or incorporated in liposomes.

25. An oligonucleotide drug according to claim 24, wherein the lipid nanoparticle or liposome is conjugated to a targeting moiety.

26. The lipid nanoparticle or liposome according to claim 24, wherein the oligonucleotide drug is further comprised of oligonucleotides selected from SEQ ID NOs 149-155, and wherein oligonucleotide modifications are comprised of locked nucleic acids, 2′-O-methyl, 2′-O-methoxyethyl, 2′-fluorine, 2′-fluoroarabinonucleic acid, and wherein nucleotide linkages are comprised of phosphorothioate or an amide.

27. The lipid nanoparticle or liposome according to claim 24 that is further comprised of proteins with a sequence selected from SEQ ID NOs 157-163.

28. An oligonucleotide drug according to claim 21, wherein the oligonucleotide sequences are inserted into an expression construct and delivered in a viral vector.

29. An expression construct according to claim 28, wherein the expression construct further contains oligonucleotide sequences selected from SEQ ID NOs 149-155.

30. A method of treating EBV infection in a patient by delivering to a patient an RNA or DNA oligonucleotide drug composed of at least two oligonucleotide sequences, wherein the oligonucleotide sequence is comprised of at least 7 contiguous nucleotides that are least 70% identical to at least two sequences selected from SEQ ID NOs 3-24 and 27-146.

31. A method according to claim 30, wherein oligonucleotide modifications are comprised of locked nucleic acids, 2′-O-methyl, 2′-O-methoxyethyl, 2′-fluorine, 2′-fluoroarabinonucleic acid, and wherein nucleotide linkages are comprised of phosphorothioate or an amide.

32. A method according to claim 31, wherein the selected oligonucleotides are delivered conjugated to a targeting moiety.

33. A method according to claim 31, wherein the selected oligonucleotides are delivered in a lipid nanoparticle or liposome.

34. A method according to claim 33, wherein the lipid nanoparticle or liposome is conjugated to a targeting moiety.

35. A method according to claim 33, wherein the lipid nanoparticle or liposome is further comprised of oligonucleotide sequences selected from SEQ ID NOs 149-155, and wherein the oligonucleotide modifications are comprised of locked nucleic acids, 2′-O-methyl, 2′-O-methoxyethyl, 2′-fluorine, 2′-fluoroarabinonucleic acid, and wherein nucleotide linkages are comprised of phosphorothioate or an amide.

36. A method according to claim 33, wherein the lipid nanoparticle or liposome is further comprised of proteins with sequences selected from SEQ ID NOs 157-163.

37. A method according to claim 30, wherein an oligonucleotide drug is delivered to a patient by inserting the oligonucleotide sequences into an expression construct within a viral vector, and wherein the virus is delivered to the patient by transduction.

38. A method according to claim 37, wherein the construct expression is regulated by an upstream tetracycline-transactivator transcription domain (SEQ ID NO 148).

39. A method according to claim 38, wherein the viral vector contains a second expression construct comprised of the sequence encoding the reverse tetracycline-transactivator (SEQ ID NO 147) and sequences selected from SEQ ID NOs 149-155, and wherein an internal ribosome entry site is inserted between the sequences.

40. A method according to claim 38, wherein the patient is administered doxycycline to induce construct expression.
